(19) United States
(12) Patent Application Publication
(10) Pub. No.: US 2004/0225004 A1
(43) Pub. Date: Nov. 11, 2004

(54) Combined use of cruciferous indoles and chelators for the treatment of papillomavirus-related conditions

(75) Inventor: Michael A. Zeligs, Boulder, CO (US)

Correspondence Address: JONES DAY, 222 EAST 41ST ST, NEW YORK, NY, 10017, US

(73) Assignee: BioResponse, LLC

(21) Appl. No.: 10/774,324

(22) Filed: Feb. 6, 2004

Related U.S. Application Data
(60) Provisional application No. 60/445,888, filed on Feb. 6, 2003.
(60) Provisional application No. 60/445,916, filed on Feb. 6, 2003.

(51) Int. Cl. A61K 31/405; A61K 31/195

(52) U.S. Cl. 514/419; 514/564

(57) ABSTRACT

Synergistic compositions and methods are disclosed for using cruciferous indoles with iron/zinc chelators in the treatment of papillomavirus-related conditions. These synergistic compositions of diindolylymethane (DIM), related trimeric derivatives, and related indole derivatives include combinations with deferoxamine, deferiprone, bipyridyl, Desferri-exochelin, picolinic acid, 3-hydroxypicolinic acid, and sodium butyrate as iron/zinc chelators. The methods described comprise more effective therapies for common cutaneous warts (verrucae) and related dysplasias of the oropharynx, genitalia, and uterine cervix.

This application claims the benefit of U.S. Provisional Application Nos. 60/445,888 and 60/445,916, both filed on Feb. 6, 2003, and which are incorporated by reference herein in their entireties.

### 1. FIELD OF THE INVENTION

The present invention includes compositions and methods for the treatment and prevention of papillomavirus-related disease, including occult infection, pre-cancerous epithelial dysplasias, and papillomavirus-related epithelial cancers. Without being bound by theory, the methods result in promotion of programmed cell death (“apoptosis”) in virally infected or damaged cells. The methods include systemic and topical combinations, result in synergistic amplification of apoptosis, and include combined compositions of indole phytochemicals, chemical iron/zinc chelators, and optionally, one or more of the iron-displacing trace element, gallium, a zinc-binding histone deacetylase inhibitor and an Epidermal Growth Factor Receptor (EGFR) antagonist. In certain embodiments, the compositions of the invention can be used in combination with radiation therapy. The induced promotion of apoptosis results in elimination of abnormal epithelial cells infected with papillomavirus, and causes resolution of papillomavirus-related lesions of skin and epithelial surfaces. The invention provides new therapeutic options for papillomavirus-related conditions.

### 2. BACKGROUND OF THE INVENTION

2.1. Epidemic Papillomavirus Infections Leads To Cancer

Papillomaviruses are small DNA viruses infecting stratified cutaneous or mucosal epithelial tissue. Prevalent in humans and animals, they are responsible for a spectrum of disease ranging from benign warts (veruccae) to malignant neoplasms. Verrucae are the most obvious sign of papillomavirus infection involving the skin. Verrucae consist of scaly rough nodules that can be found on any skin surface. They are benign proliferations of epithelial cells most commonly involving the hands and soles of the feet. Verrucae spread locally to develop in sites adjacent to viral inoculation. Spread is also related to immune status, and verrucae are therefore more common in children and immune-impaired adults. Besides verrucae, papillomavirus infection often results in oral-genital manifestations. Oral-genital manifestations include oropharyngeal papillomas and dysplasia, peri-anal verrucae, virus-related papillomas and dysplasia, vaginal papillomas and dysplasia, and uterine cervical papilloma virus-related papilloma and dysplasia. Papillomavirus induced dysplasia progresses unpredictably to intra-epithelial neoplasia and subsequently to a number of types of cancer. The presence of papilloma virus-specific DNA in cancerous tumor tissue and the absence in normal surrounding tissue has now been used to establish the contribution of papilloma virus infection to the occurrence and progression of certain types of cancer including non-melanoma skin cancer, squamous cell head and neck cancer, esophageal cancer, anal cancer, cervical cancer, and prostate cancer.

2.2. Papillomaviruses Disrupt Epithelial Apoptosis

The papillomavirus family now includes over 100 viral genotypes with different subtypes more prone to cause disease involving specific epithelial surfaces. The common mechanism of action of the viruses is to induce hyperproliferation of basal cell types. This mode of action at the molecular level involves specific growth signals from virus-derived oncoproteins (e.g., E5, E6 and E7) which disrupt normal cell function. These proteins override normal cell-cycle signals and result in the suspension of normal “programmed cell-death”, termed “apoptosis”, in infected epithelial cells. The papillomavirus E5 protein activates an anti-apoptotic pathway mediated by Epidermal Growth Factor (EGF) causing the persistence of virally infected cells and making such cells resistant to the protective apoptotic response following exposure to ultraviolet light (UVA, UVB, UVC) or other radiation including X rays. This causes infected cells to persist and undergo abnormal, unscheduled cell-division while harboring viral DNA. This unscheduled growth results in characteristic dysplasia, a pre-cancerous change in cell appearance and behavior observable with routine microscopic examination. Dysplasia of the uterine cervix in women, diagnosed by the Papinicolou Cervical Smear (Pap Test), is a common condition that is linked to the presence of papillomavirus and a known cause of cervical cancer. (Walboomers J M, Jacobs M V, Manos M M, Bosch F X, Kummer J A, Shah K V, Snijders P J, Peto J, Meijer C J and Munoz N, Human papillomavirus is a necessary cause of invasive cervical cancer worldwide. J Pathol. 1999 September;189(1):12-9). Recently, the presence of papillomavirus DNA has been detected in a variety of other epithelial cancers, including head and neck cancer (Gillison M L and Shah K V, Human papillomavirus-associated head and neck squamous cell carcinoma: mounting evidence for an etiologic role for human papillomavirus in a subset of head and neck cancers. Curr Opin Oncol. 2001 May;13(3):183-8), esophageal cancer (Hasegawa M, Ohoka I, Yamazaki K, Hanami K, Sugano I, Nagao T, Asoh A, Wada N, Nagao K and Ishida Y, Expression of p21/WAF-1, status of apoptosis and p53 mutation in esophageal squamous cell carcinoma with HPV infection. Pathol Int. 2002 July;52(7):442-50), and squamous cell cancer of the skin (Harwood C A and Proby C M, Human papillomaviruses and non-melanoma skin cancer. Curr Opin Infect Dis. 2002 April;15(2):101-14). The presence of the virus has now been detected in men as well, establishing prostate gland epithelial tissue as a site of asymptomatic viral infection, and explaining the efficient transmission of papillomavirus as a sexually transmitted disease (Zambrano A, Kalantari M, Simoneau A, Jensen J L, Villarreal L P, Detection of human polyomaviruses and papillomaviruses in prostatic tissue reveals the prostate as a habitat for multiple viral infections. Prostate. 2002 Dec. 1;53(4):263-76). The presence of human papillomavirus (HPV) DNA has been demonstrated in prostate cancer at rates greater than that seen in benign prostatic disease (Serth J, Panitz F, Paeslack U, Kuczyk M A and Jonas U, Increased levels of human papillomavirus type 16 DNA in a subset of prostate cancers. Cancer Res. 1999 Feb. 15; 59(4): 823-5). Other cancers established as papillomavirus-related include, vulvar cancer, anal cancer, penile cancer, oropharyngeal cancer, and conjunctival cancer.

2.3. Oral Indole-3-Carbinol(I3C) Is A Source Of Anti-Papillomavirus Activity

Cruciferous vegetables contain a family of plant protective compounds called glucosinolates which give rise to active compounds with indole rings exemplified by indole-3-carbinol (I3C). The action of I3C in cell culture models has been associated with the promotion of apoptosis in a variety of cell types (Chinni S R, Li Y, Upadhyay S, Koppolu P K and Sarkar F H, Indole-3-carbinol (I3C) induced cell growth inhibition, G1 cell cycle arrest and apoptosis in prostate cancer cells. Oncogene. 2001 May 24;20(23):2927-36). In animal models, I3C administration has been associated with the prevention of HPV-related cervical dysplasia (Jin L. et al., Indole-3-carbinol prevents cervical cancer in human papilloma virus type 16 (HPV16) transgenic mice, Cancer Res. 1999, 59(16):3991-7). Preliminary human testing of I3C in cervical dysplasia has been associated with partial improvement in about 50% of women treated for 3 months (Bell M C, Crowley-Nowick P, Bradlow H L, Sepkovic D W, Schmidt-Grimminger D, Howell P, Mayeaux E J, Tucker A, Turbat-Herrera E A and Mathis J M, Placebo-controlled trial of indole-3-carbinol in the treatment of CIN. Gynecol Oncol. 2000 August;78(2):123-9).

However, I3C is highly unstable in water and acid. When given orally, I3C generates a number of gastric reaction products with a variety of biologic actions (De Kruif Calif., Marsman J W, Venekamp J C et al., Structure elucidation of acid reaction products of indole-3-carbinol: detection in vivo and enzyme induction in vitro. Chem Biol Interact 1991; 80(3):303-15). These products are highly enzyme inducing and associated with both the inactivation and activation of carcinogens. As such, the use of I3C has been associated with both the prevention and promotion of experimental cancers. In addition, unwanted enzyme induction by I3C reaction products following oral I3C use may alter the metabolism of other drugs, steroid hormones, and contraceptives raising safety concerns. Reports of adverse side effects with I3C use at higher doses in animals and in individuals with papillomavirus infection and respiratory tract papillomas have discouraged further clinical testing of I3C in cervical dysplasia (Rosen, C.A., Woodson, G. E. et al., Preliminary results of the use of indole-3-carbinol for recurrent respiratory papillomatosis. Otolaryngology Head Neck Surgery 1998, 118:810-5). Furthermore, I3C's use is associated with a number of safety concerns due to its enzyme-inducing and reproductive-toxic actions (Dashwood R H, Indole-3-carbinol: anticarcinogen or tumor promoter in brassica vegetables? Chem Biol Interact. 1998 Mar 12, 110(1-2):1-5; Gao X, Petroff B K, Oluola O, Georg G, Terranova P F and Rozman K K, Endocrine disruption by indole-3-carbinol and tamoxifen: blockage of ovulation. Toxicol Appl Pharmacol. 2002 Sep. 15;183(3):179-88).

2.4. Diindolylmethane (DIM) May Be An I3C Derivative Active Against Papillomavirus-related Conditions

One prominent product derived from I3C, but also present in cruciferous plants is diindolylmethane (DIM). Once formed, DIM is stable in acid, and less enzyme inducing than other I3C products (Bradfield C A and Bjeldanes L F, Structure-activity relationships of dietary indoles: a proposed mechanism of action as modifiers of xenobiotic metabolism. J Toxicol Environ Health. 1987;21(3):311-23). In cell culture, DIM has been shown to have apoptosis promoting effects in both estrogen-dependent and independent breast cancer cells (Hong C, Firestone G L and Bjeldanes L F, Bc1-2 family-mediated apoptotic effects of 3,3′-diindolylmethane (DIM) in human breast cancer cells. Biochem Pharmacol. 2002 Mar. 15;63(6):1085-97).

In animals, orally administered DIM inhibits the growth of certain chemically induced forms of breast cancer (Chen I et al., Aryl hydrocarbon receptor-mediated antiestrogenic and antitumorigenic activity of Diindolylmethane. Carcinogenesis 1998, 19(9):1631-9). Recently, DIM has been shown to specifically induce apoptosis in papillomavirus altered cervical cancer cell lines (Chen D Z, Qi M, Auborn K J and Carter T H, Indole-3-carbinol and diindolylmethane induce apoptosis of human cervical cancer cells and in murine HPV16-transgenic preneoplastic cervical epithelium. J Nutr. 2001 December;131(12):3294-302). This cell culture work demonstrated that DIM was more active than I3C in inducing markers of apotosis. However, the activity of DIM required a concentration of 50 micromolar, far in excess of the levels achievable in vivo. Unlike the experimental uses of I3C in animals and humans, there have been no reports on the usefulness of DIM in the treatment of papillomavirus-related conditions in vivo.

2.5. Iron And Zinc Are Regulators Of Cell Growth And Apoptosis

Iron and Zinc are absorbed from the diet as nutritional substances in their ionized, soluble state. They are incorporated into biomolecules and enyzymes where they serve as catalytic sites for essential biochemical reactions.

Iron deficiency, sensed by the cell, is linked to the natural process of apoptosis or “programmed cell death” (Fukuchi K, Tomoyasu S, Tsuruoka N and Gomi K, Iron deprivation-induced apoptosis in HL-60 cells. FEBS Lett. 1994 Aug. 15;350(1):139-42). Iron excess is a signal for greater intracellular production of iron-binding transferin protein which protects the cell from free-iron generated free radical electrons and associated molecular damage. Once intracellular, iron/zinc chelators disrupt iron and zinc from their metalloenzymes and expose free iron and zinc cations for uncontrolled reactions. This results in free radical related oxidative stress and deficient activity of metalloenzymes. Nuclear DNA is protected from oxidative damage by surrounding histone proteins. Intra-nuclear zinc is required for the regulatory activity of histone protein acetylation and deacetylation due to the fact that histone deacetylase enzymes utilize zinc in their active sites. Binding of zinc by chelators and zinc-interacting inhibitors of histone deacetylase enzymes results in oxidant stress, DNA damage, and apoptosis.

2.6. Iron and Zinc Chelators Can Act to Promote Apoptosis

When cells are made iron or zinc deficient through the iron/zinc sequestering activity of iron/zinc chelator substances, normal cell growth is disrupted. In cell culture studies of certain cancer cells the use of iron/zinc chelators has been associated with the promotion of apoptosis. However, the activity of iron/zinc chelators in vitro has required high levels not readily achieved in vivo. Iron and zinc sequestration through the use of iron and zinc chelating substances has been investigated as a means of inhibiting cancerous cell growth (Gao J and Richardson D R, The potential of iron chelators of the pyridoxal isonicotinoyl hydrazone class as effective antiproliferative agents, IV: The mechanisms involved in inhibiting cell-cycle progression. Blood. 2001 Aug. 1;98(3):842-50). Similarly, zinc chelators and zinc-binding histone dacetylase inhibitors have been shown to promote apoptosis in cancer cells and in vivo animal models (Marks P A, Richon V M and Rifkind R A, Histone deacetylase inhibitors: inducers of differentiation or apoptosis of transformed cells. J Natl Cancer Inst. 2000 Aug. 2; 92(15):1210-6). Sodium butyrate is a nontoxic short chain fatty acid which interacts with nuclear zinc, has shown growth inhibitory activity in a number of cancer cell types, and acts through promotion of apoptosis (Terao Y, Nishida J, Horiuchi S, Rong F, Ueoka Y, Matsuda T, Kato H, Furugen Y, Yoshida K, Kato K and Wake N, Sodium butyrate induces growth arrest and senescence-like phenotypes in gynecologic cancer cells. Int J Cancer 2001 Oct. 15; 94(2): 257-67). The limitations of this theoretical approach to cancer treatment have to do with the limited selectivity of iron/zinc chelators for cancerous cells compared to normal cells, the high dose requirements for effective local tissue concentrations, and general toxicity of metal chelators in biologic systems.

Though some temporary improvement in advanced cancers, such as neuroblastoma, has been observed with the use of an iron chelator, no durable control of cancer in vivo has resulted. When tested in vivo in the case of Kaposi's Sarcoma, a cancer of epithelial tissue, both animals and humans showed a paradoxical promotion of tumor growth (Simonart T, Boelaert J R, Andrei G, van den Oord J J, Degraef C, Hermans P, Noel J C, Van Vooren J P, Heenen M, De Clercq E and Snoeck R, Desferrioxamine enhances AIDS-associated Kaposi's sarcoma tumor development in a xenograft model. Int J Cancer 2002 Jul. 10;100(2):140-3). This indicates that the action of chelators on cancer cells in vivo, particularly epithelial cells and epithelial cancers, is unpredictable, may not reflect in vitro effects, and alone, has not been shown to be adequate or efficacous therapy.

2.7. Iron/zinc Chelators Demonstrate Antiviral Activity In Cell Culture

A study of cell membrane permeable iron/zinc chelators in cell culture has demonstrated arrest of viral replication and induction of apoptosis (Fernandez-Pol J A, Klos D J and Hamilton P D, Antiviral, cytotoxic and apoptotic activities of picolinic acid on human immunodeficiency virus-1 and human herpes simplex virus-2 infected cells. Anticancer Res. 2001 November-December;21(6A):3773-6). This work included use of picolinic acid in human immunodeficiency virus-1 (HIV-1) and herpes simplex virus-2 (HSV-2) infected cells. These authors demonstrated that slowing of growth and diminished viability of virus infected skin cells requires a 3,000 micromolar concentration of picolinic acid. More potent anti-viral activity was subsequently demonstrated for Bypyridyl (2,2′-Bipyridyl) where a study of the Vaccinia virus was conducted in monkey kidney cells (Romeo A M, Christen L, Niles E G and Kosman D J, Intracellular chelation of iron by bipyridyl inhibits DNA virus replication: ribonucleotide reductase maturation as a probe of intracellular iron pools. J. Biol. Chem. 2001 Jun. 29;276(26):24301-8). Concentrations of Bypyridyl in the range of 60-80 micromolar were shown to inhibit Vaccinia virus replication and inhibition of iron dependent ribonucleotide reductase (RR). RR is a critical enzyme reducing ribonuceotides to desoxyribonucleotides for DNA synthesis, cell division, and viral replication.

2.8. Gallium Displaces Iron/zinc

A related approach to iron/zinc depletion by iron/zinc chelators to induce growth inhibition involves using certain trace metals, similar to iron/zinc in molecular weight and ionic charge. This applies to the trace element, gallium. Instead of inactivating enzymes through the formation of molecular complexes as chelators do, gallium replaces iron/zinc as an inactive occupant of its enzymatic sites of action. As with iron/zinc chelators, treatment of cells with gallium salts results in inhibition of ribonucleotide reductase (RR) and tumor cell growth arrest. When used as a component of cancer therapy, intravenous gallium replaces iron bound to transferin in and outside cells and causes anemia and other systemic side effects (Apseloff G, Therapeutic uses of gallium nitrate: past, present, and future. Am J Ther. 1999 November;6(6):327-39). Gallium-67 (Ga-67), an unstable radioactive isotope of Gallium, is known to preferentially accumulate in a variety of abnormal tissue including some cancers. Intravenous Ga-67 has been used for decades as means of visualizing the presence of tumor tissue using radionucleotide scanning (Scintography).

2.9. Therapeutic Applications Of Diindolylmethane (DIM), Iron/zinc Chelators, And Gallium

Earlier investigations of DIM resulted in U.S. Pat. No. 5,948,808 providing for a method of treating estrogen-dependent tumors. U.S. Pat. No. 6,001,868 discloses other derivatives of I3C as a method to inhibit tumor cell growth, but specifically excludes Diindolylmethane. The use of DIM and related 2-(indol-3-ylmethyl)-3,3′-diindolylmethane [also written: 2-(Indol-3-ylmethyl)-indol-3-yl]indol-3-ylmethane] (LTR) for the therapy of HPV related conditions has been described by the present inventor in co-pending U.S. patent application Ser. No. 10/117,288. These uses require high doses of DIM and a 6-8 week treatment period. Combined uses of DIM with immune potentiating steroid substances like dehydroepiandrosterone (DHEA) and pregnenolone are also disclosed.

Iron/zinc chelators have been demonstrated to be potentially useful in the control of cancer cell growth in vitro. These have included traditional iron chelators like Desferrioxamine (DFO), but recently greater activity has been seen when more cell membrane permeable compounds are used, for example, the control of breast cancer cell growth with exochelins, described in U.S. Pat. No. 6,335,443, and with N,N′-bis(2-hydroxybenzyl) ethylenediamine-N,N′-diacetic acid (HBED) disclosed in U.S. Pat. No. 6,242,492.

Topical uses of picolinic acid and fusaric acid have been described for skin lesions characteristic of HPV. These uses for HPV related conditions have been described in U.S. Pat. Nos. 5,767,135 and 6,410,570. In these treatments, therapeutic responses of cutaneous warts required a solution or ointment of 10-20% concentration of picolinic acid and a 6 week to 8 week duration of therapy for the response to therapy.

Apart from vaccines which may induce resistance to initial infection with papillomaviruses, various other methods of treating warts and established papillomavirus conditions have been suggested, for example, in U.S. Pat. No. 6,406,706 (Sandlewood oil), U.S. Pat. No. 6,468,557 (corticosteroids and L-lysine), and U.S. Pat. No. 6,432,926 (papillomavirus proteins).

In addition, allegedly immune potentiating therapies for papillomavirus-related disease have included the local application of mumps vaccine, the use of cidofinir systemically and locally to HPV lesions, local and systemic uses of extracts from Aloe Vera, and topical uses of skin irritants including, salicylic acid, podophylox (Condylox, Occassen Dermatologics) and imiquimod (Aldara Cream, 3M Pharmaceuticals).

2.10. More Effective Anti-Papillomavirus Treatments Are Needed

Both DIM and certain Iron/Zinc chelators show promise as potential anti-cancer and anti-viral compounds. However, both modalities involve limitations due to their physico-chemical characteristics. Chelator therapy for virus-related disease has limitations due to high concentrations required for minimally effective dose, lack of specificity of chelator substances for infected versus normal cells, systemic toxicity of chelators, and damage by chelators to normal bystander cells in various tissues. The basis of chelator toxicity includes the disruption of essential metal dependent enzyme activity. No controlled clinical studies have yet demonstrated success with chelator therapy alone in virus-related conditions. DIM is a highly insoluble substance demonstrating negligible dissolution in water and oil. Its use as a therapeutic requires special consideration as to its formulation for adequate absorption and skin penetration to achieve minimally effective concentrations.

Safer, more consistently effective treatments are needed for papillomavirus-related conditions, including cervical dysplasia, and common warts. These needs extend to better options for prevention and treatment of papilloma virus related cancers including non-melanoma skin cancer, conjunctival cancer, oropharyngeal cancer, esophageal cancer, anal cancer, cervical cancer, and prostate cancer. Based on the inconsistent response to current therapies, the requirement for prolonged and painful treatment intervals, and potential toxicity of existing therapies, new, less-invasive, more rapid acting, and more consistently effective therapies for papillomavirus-related conditions are needed.

Ideally, new therapies will prove effective against the varied spectrum of papillomavirus related disease.

### 3. SUMMARY OF THE INVENTION

Synergistic compositions comprising one or more cruciferous indoles and one or more metal chelators for the treatment of papillomavirus-related conditions and methods of treating papillomavirus-related conditions by administering one or more cruciferous indoles and one or more metal chelators are provided. In a particular embodiment, the one or more cruciferous indoles of the invention are selected from the group consisting of I3C, DIM, the related trimeric derivative, 2-(indol-3-ylmethyl)-3,3′-diindolylmethane (LTR), and related hydroxylated and methoxylated DIM metabolites. In particular embodiment, the one or more metal chelators of the invention are selected from the group consisting of Desferrioxamine (DFO) (Novartis, Basel, Switzerland), and 1,2-Dimethyl-3-hydroxypyrid-4-one (deferiprone [L1], Apotex, Toronto), picolinic acid, and sodium butyrate. In certain embodiments, one or more cruciferous indoles and one or more metal chelators is administered, either in the same composition or separately, with one or more of the following: a zinc-binding histone deacetylase inhibitor, gallium or an EGFR (epidermal growth factor receptor) antagonist. In another embodiment, a method or composition of the invention is employed in conjunction with radiation therapy.

In a particular embodiment, the papillomavirus-related condition is common cutaneous warts (verrucae) often involving the hands and the feet. In addition, the present invention provides useful methods for the treatment of papillomavirus-related oral-genital papillomavirus infections, and for uterine cervical papillomavirus-related conditions, including cervical dysplasia and papillomavirus-related cancer.

In a particular embodiment, the cruciferous indole, the chelator, and optionally one or more of a zinc-binding histone deacetylase inhibitor, gallium or an EGFR antagonist, are administered simultaneously. In another embodiment, the cruciferous indole, chelator and optionally one or more of a zinc-binding histone deacetylase inhibitor, gallium and an EGFR antagonist are administered within a short time of one another, for example, 30 seconds, 1 minute, 5 minutes, 15 minutes, 30 minutes, 1 hour, 4 hours, 8 hours, 12 hours or 24 hours of one another.

In one embodiment of the present invention, the one or more cruciferous indoles, for example, DIM, in combination with one or more iron/zinc chelators and the trace element, gallium, are used to treat papillomavirus infected epithelia administered locally. This includes the use of topical combined formulations which may further comprise penetration enhancers, pH adjusters, and osmotic agents. Intra-lesional injection therapy as described permits different combinations of cruciferous indoles, chelators, gallium, and pH adjusters. Because of their significant systemic safety relative to most chelators, oral cruciferous indoles, for example, DIM, can be used in conjunction with topical application of cruciferous indoles, chelators, sodium butyrate, and gallium combinations. Various specialized formulations of the combinations, including the use of liposomes to encapsulate cruciferous indoles with chelators, and specialized penetration enhancers are designed for particular epithelial surfaces, including skin, vaginal, rectal, ocular and oral mucosa. These specialized formulations include uses as part of topical, papillomavirus-preventive contraceptives when the formulated components of the present invention are combined with established spermicides. Special encapsulated, non-absorbed oral formulations of, e.g., cruciferous indoles, chelators and gallium can also be used to target esophageal, colonic and rectal epithelia. This offers an approach to the treatment of esophageal, rectal and anal dysplasia and other papillomavirus-related disease involving the gastrointestinal tract.

Methods according to the invention include a method of treating a papillomavirus related epithelial disorder comprising administering to a subject in need thereof a therapeutically effective amount of an iron/zinc chelator and a cruciferous indole. In particular embodiments, the chelator and indole are administered simultaneously, or the chelator and indole are administered within a short time of one another. In another embodiment, the indole is administered orally. In a particular embodiment, the amount of the indole administered is lower than that which is therapeutically effective when the indole is administered in the absence of the chelator. In a particular embodiment, the amount of the chelator is lower than that which is therapeutically effective when the chelator is administered in the absence of the indole. In another embodiment, both the amount of the chelator and indole are lower than that which is therapeutically effective when the chelator or indole is administered in the absence of the other. In a preferred embodiment, the iron/zinc chelator and the indole act synergistically. In another embodiment, the method comprises the further administration of a therapeutically effective amount of a gallium salt, gallium isotope, zinc-binding histone deacetylase inhibitor or epidermal growth factor receptor antagonist. When a gallium salt is administered, preferably the chelator has an affinity for gallium and an affinity for iron/zinc, and wherein the affinity for gallium is less than the affinity for iron/zinc. In a further embodiment, the combination of indole and iron/zinc chelator and optionally one or more of a gallium salt, gallium isotope, zinc-binding histone deacetylase inhibitor or epidermal growth factor receptor antagonist, are administered in conjunction with a radiation therapy regimen sufficient to treat a papillomavirus-related disease. In a preferred embodiment, topical ultraviolet light or site directed ionizing radiation (X-rays) is used.

In particular embodiments, the papillomavirus related epithelial disorder treated according to the method of the invention is oral-genital human papilloma virus infection, oropharyngeal human papilloma virus-related papillomas and dysplasia, peri-anal human papilloma virus-related papilloma and dysplasia, vaginal human papilloma virus-related papilloma and dysplasia, uterine cervical human papilloma virus-related papilloma and dysplasia, skin-related human papilloma virus infection (warts or verrucae), human papilloma virus-related cancer, basal cell carinoma of the skin, carcinoma of the uterine cervix, carcinoma of the uterine endometrium, carcinoma of the colon, carcinoma of the anus, oropharyngeal carcinoma, esophageal carcinoma, prostate carcinoma or an opthalmic papillomvirus-related condition. Treatments according to the invention are also directed at less common papillomavirus related skin diseases including Epidermodyplasia Verruciformis, giant condyloma acuminatum, called the Buschke-Lowenstein tumor, involving the soles of the feet, and Bowenoid papulosis, involving the external male and female genitalia.

The invention further provides pharmaceutical compositions, for example, a pharmaceutical composition comprising a therapeutically effective amount of the combination of an iron/zinc chelator and a cruciferous indole. In particular embodiments, the composition is formulated for oral administration, topical, or intravenous administration. In particular embodiments, the amount of the indole in the composition is lower than that which is therapeutically effective when the indole is administered in the absence of the chelator. In particular embodiments, the amount of the chelator is lower than that which is therapeutically effective when the chelator is administered in the absence of the indole. In a particular embodiment, both the amount of the indole and the chelator are lower than that which are therapeutically effective when the chelator or indole are administered in the absence of the other. Preferably, the compositions of the invention comprise a synergistic combination of cruciferous indole and chelator. In a particular embodiment, the composition of the invention further comprises one or more of a therapeutically effective amount of gallium, a gallium salt or isotope, a zinc-binding histone deacetylase inhibitor or an EGFR antagonist. When the composition comprises a gallium salt, preferably the chelator has an affinity for gallium and an affinity for iron/zinc, and wherein the affinity for gallium is less than the affinity for iron/zinc.

### 4. BRIEF DESCRIPTION OF DRAWINGS

FIG. 1 is a bar chart depicting the effects of silybin (SY), diindolylmethane (DIM) and diidolylmethane plus silybin (DIM+SY) to induce cytotoxity in C33A cells. “*” indicates synergism.

FIG. 2 is a bar chart depicting the effects of silybin (SY), diindolylmethane (DIM), and diidolylmethane plus silybin (DIM+SY) to induce cytotoxity in CaSki cells.

FIG. 3 is a bar chart depicting the effects of silybin (SY), diindolylmethane (DIM), diindolylmethane plus silybin (DIM+SY), sodium butyrate (BU), and diindolylmethane plus sodium butyrate (DIM+BU) to induce cytotoxity in CaSki cells. “*” indicates synergism.

### 5. DETAILED DESCRIPTION OF THE INVENTION

As used herein, an “iron/zinc” chelator refers to a chelator which has affinity for iron, zinc or both. An iron/zinc chelator which has affinity for both iron and zinc need not have the same affinity for both.

The present invention is based upon the observation that living cells are sensitive to iron/zinc status and can respond to induced changes in trace metal activity with cell death. Papillomavirus infected cells are similarly sensitive to both alterations of iron/zinc activity and the presence of cruciferous indoles, for example, DIM or its active metabolites. Without being bound by theory, the intracellular presence of cruciferous indoles combined with altered intracellular activity of iron and/or zinc reverses the effects of growth promoting papillomavirus oncoproteins and forces dividing cells back into programmed cell death, or “apoptosis”.

Apoptosis is a primary biologic defense in response to viral infection and pre-cancerous cellular damage. Creation of an altered cellular iron/zinc status is recognized as a potential approach to the treatment and selective elimination of certain cancer cells (Gao J and Richardson D R, The potential of iron chelators of the pyridoxal isonicotinoyl hydrazone class as effective antiproliferative agents, IV: The mechanisms involved in inhibiting cell-cycle progression. Blood 2001 Aug. 1; 98:(3):842-50). Prior work by the present inventor has demonstrated that administration of DIM and related cruciferous indoles results in the spontaneous remission, resolution and healing of common cutaneous warts (verrucae) and related oral-genital papillomvirus (HPV) infections. These effects of DIM are concentration dependent and require significant periods of time for successful therapeutic response. Similarly, iron/zinc chelators have been observed to inhibit growth in some virally infected cells (Romeo A M, Christen L, Niles E G and Kosman D J, Intracellular chelation of iron by bipyridyl inhibits DNA virus replication: ribonucleotide reductase maturation as a probe of intracellular iron pools. J. Biol. Chem. 2001 Jun. 29;276(26):24301-8). But, as with DIM therapy, the use of iron/zinc chelators requires high concentrations and prolonged therapy.

Combined, synergistic administration of cruciferous indoles with certain chelators for the treatment of papillomavirus-related conditions are now provided. The combined administration of a cruciferous indole and a metal chelator exhibits greater than additive effect, i.e., the combination is synergistic. This complementary, synergistic action includes promotion of apoptosis seen with the combinations. In addition, gallium is used in certain combinations as an iron/zinc displacing trace element which is used in combined therapy to further potentiate combinations of DIM and iron/zinc chelators.

Methods of use rely on the unexpected synergy of combining one or more cruciferous indoles, preferably DIM, with one or more chelators, preferably iron/zinc chelators, in treating or preventing papillomavirus-related conditions.

The chemical structure of DIM is as follows:

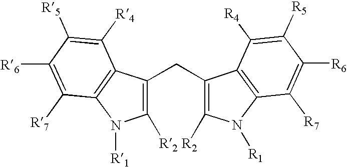

The cruciferous indoles useful in the methods of the invention include DIM, I3C, the related linear DIM trimer (2-(indol-3-ylmethyl)-3,3′-diindolylmethane [also written: 2-(Indol-3-ylmethyl)-indol-3-yl]indol-3-ylmethane] (LTR), active hydroxylated metabolites of DIM (R′₅ hydroxy-DIM, R′₅ methoxy-DIM, R₅ hydroxy-DIM, R₅ methoxy-DIM, R′₅ R₅ dihydroxy-DIM, R′₅ R₅ dimethoxy-DIM, R′₅ R′₄ dihydroxy-DIM, R₅ R₄ dihydroxy-DIM, R′₅ methoxy R′₄ hydroxy-DIM, and R₅ methoxy R₄ hydroxy-DIM). Active DIM derivatives including imidazolelyl-3,3′-diindolylmethane, including nitro substituted imidazolelyl-3,3′-diindolylmethanes, and Stanford Research Institute DIM derivative SR13668 (SRI Inc., Menlo Park, Calif.) are also useful, as well as 2-hydroxy and 2-methoxyestradiol which promote apoptosis and can be used alone or in conjunction with the above cruciferous indoles, DIM and its active metabolites.

Chelators for use according to the invention include, but are not limited to, iron/zinc chelators (e.g., Desferrioxamine (DFO) (Desferal, Novartis, Basel, Switzerland)), 3,5,7,-trihydroxy-2-[3-(4-hydroxy-3-methoxyphenil)-2-hydroxymethyl-1,4-benxodioxan-6-il]-chronan-4-one (Silybin), ethylenediametetraacetic acid [EDTA], and ethylenetriaminepentaacetic acid [DTPA], 1,2-Dimethyl-3-hydroxypyrid-4-one (deferiprone, Ferriprox [L1]), other hydroxypyridin-4-ones (U.S. Pat. No. 6,335,353, incorporated by reference herein in its entirety), Desferri-Exochelin [DFE 772SM] (Keystone Biomedical, Inc.), N,N′-bis(2-hydroxybenzyl)ethylenediamine-N,N′-diacetic acid (HBED), picolinic acid, 3-hydroxypicolinic acid, and Fusaric acid, topical 2-furildioxime (FDO, Eastman Kodak, Rochester, N.Y.), 2,2′-bypryidyl (dipyridine [bipryidyl]), its derivative, 2,2′-bipyridyl-6-carbothioamide (BPYTA), and 1,10-Phenanthroline), zinc-binding Sodium Butyrate (Butyric acid sodium salt), Tributyrin, butanoic acid, 1,2,3-propanetriyl ester, an orally active butyric acid prodrug, and suberoylanilide hydroxamic acid (SAHA), a butyric acid related compound. Other chelators useful in the methods and compositions of the invention are described in U.S. Pat. No. 5,834,492, which is incorporated herein in its entirety.

Alternatively, the trace element gallium is used, for example, in the form of gallium nitrate or sulfate, in conjunction with certain of the iron/zinc chelators. Preferably, when gallium is used, the chelator or chelators used have a higher affinity for iron/zinc than they do gallium. Cruciferous indoles, e.g., DIM, chelators, and gallium are applied locally to skin or mucous membranes infected by papillomavirus. Local use includes topical application and intra-lesional injection. Intravenous uses includes infusions of Gallium nitrate (Ganite®, NCI, Bethesda, Md.) or radioactive Gallium-67 isotope solutions (Gallium-67 Citrate, Cardinal Health, Denver, Colo.) used with oral or intravenous DIM.

In another embodiment, one or more cruciferous indoles and one or more iron/zinc chelators are combined with an epidermal growth factor receptor antagonist. Representative EGFR antagonists include, but are not limited to, IRESSA® (Gefitinib [ZD1839], 4-Quinazolinamine, N-(3-chloro-4-fluorophenyl)-7-methoxy-6-[3-4-morpholin) propoxy], Astra Zeneca, UK), CI 1033 [Parke-Davis Pharmaceutical Research (Ann Arbor, Mich.)], a quinazoline tyrosine kinase inhibitor different from Iressa, and PKI 166 [Novartis Pharma, AG (Basel)], a non-quinazoline EGFR antagonist. Gallium, preferably, the Gallium-67 isotope, can also be used in such combinations.

In yet another embodiment, one or more cruciferous indoles and one or more iron/zinc chelators, and optionally, one or more of a zinc-binding histone deacetylase inhibitor, gallium or an EGFR antagonist, can be administered in conjunction with radiation therapy. Radiation treatment plans are described herein and in U.S. Pat. Nos. 6,477,229, 6,144,875 and 5,207,223, all of which are expressly incorporated herein by reference in their entireties. One of skill in the art would be able to modify the disclosed treatment plans to allow for the therapeutic contribution of DIM and an iron/zinc chelator or other compositions disclosed in the present invention.

For the purposes of this invention chelators are classified according to their membrane permeation characteristics and as to their selective affinity for iron, zinc and gallium. These physicochemical characteristics determine the basis for optimal anti-papillomavirus activity during combined use with DIM.

Class I Chelators are large, ionically charged molecules at relevant pH, which traverse cell membrane only to a minor degree. Some cell penetration occurs through the process of endocytosis, but little to no permeation of cell membranes occurs by osmotic diffusion. Chelators in Class I are exemplified by desferrioxamine-B [DFO] (Desferal, Novartis) a siderophore class chelator of high molecular weight. Siderophores are bacteria derived compounds which selectively bind trace metals from their environment allowing the bacteria to compete for essential metals. A second example of Class I Chelators are the aminocarboxylate agents; ethylenediametetraacetic acid [EDTA], and ethylenetriaminepentaacetic acid [DTPA]. Thirdly, Silybin (3,5,7,-trihydroxy-2-[3-(4-hydroxy-3-methoxyphenil)-2-hydroxymethyl-1,4-benxodioxan-6-il]-chronan-4-one) is a naturally occurring flavolignan isolated from the fruits of Silibum marianum (Milk Thistle) compound which has demonstrated specific iron chelating activity greater than EDTA and DFO (Borsari M, Gabbi C, Ghelfi F, Grandi R, Saladini M, Severi S and Borella F, Silybin, a new iron-chelating agent. J Inorg Biochem. 2001 June;85(2-3):123-9).

Class II Chelators are smaller molecules than Class I chelators and demonstrate good penetration of cell membranes due to physico-chemical characteristics which include neutral charge, good lipid solubility (high octanol/water partition coefficient), and neutral charge. They typically have high affinity for both iron/zinc and gallium and are therefore non-specific chelators. Class II Chelators are exemplified by deferiprone [L1] (Ferriprox, 1,2-Dimethyl-3hydroxy-pyridin-4-one), a hydroxypyridinone chelator, and other hydroxypyridin-4-ones (U.S. Pat. No. 6,335,353, incorporated by reference herein in its entirety). A second example of a Class II Chelator is the hexadentate phenolic aminocarboxylate iron chelator, N,N′-bis(2-hydroxybenzyl) ethylenediamine-N,N′-diacetic acid (HBED) and its monosodium salt (NaHBED). A third example of a class II chelator is exochelin, Desferri-Exochelin [DFE 772SM] (Keystone Biomedical, Inc. [U.S. Pat. No. 6,335,443, incorporated by reference herein in its entirety]), a synthetic, membrane permeable bacterial siderophore derived from Mycobacterium tuberulosis. Other examples of a Class II Chelators are picolinic acid, 3-hydroxypicolinic acid, and fusaric acid. Picolinic acid is a natural metabolite of the essential amino acid tryptophan. Fusaric acid is a derivative of picolinic acid. Dihydroxybenzoic acid (2,3-dihydroxybenzoic acid) is a low affinity and non-toxic Class II Chelator. A final example of a Class II chelator is the triazole chelator, N,N′-bis(2-hydroxybenzyl)ethylenediamine-N,N′-diacetic acid (HBED)(U.S. Pat. No. 6,242,492, incorporated by reference herein in its entirety).

Using certain Class I chelators in conjunction with Class II chelators has been found in the present invention to provide amplified anti-papillomavirus activity in conjunction with DIM. Consistent with their relative iron/zinc binding affinities co-administering Silybin (Class I) with L1 (Class II) provides a therapeutic advantage. Similarly the combination of certain, compatible Class II chelators have been found to offer a treatment advantage as with the combination of HBED with L1.

Class III Chelators are membrane permeable compounds which demonstrate significant differential affinity for iron/zinc versus gallium. This class of chelators is exemplified by the didpyridine, bipryidyl (2,2′-bypryidyl), and its derivative, 2,2′-bipyridyl-6-carbothioamide (BPYTA). Similar in activity is 1,10-Phenanthroline.

Class III chelators demonstrate differential attraction to iron/zinc which is significantly greater than their affinity for gallium. As a result, the affinity constant for Class III Chelators, expressed as the “Log cumulative stability constant”, is 2 times higher for Fe(III), Fe(II), and Zn(II) than it is for Ga(III)(Martell AE and Smith RM. Critical Stability Constants. Vols. 1-6. London: Plenum Press, 1974-1989).

Class III Chelators act as carriers for gallium, delivering gallium to intracellular sites, then shift to associate with intracellular iron/zinc, and release bound gallium within the cell. Using Class III Chelators in conjunction with gallium has been found in the present invention to add to the iron/zinc disrupting potential of co-administered gallium and further promote apoptosis in combination with DIM.

The following table summarizes the properties of preferred iron/zinc chelators for use according to the present invention with respect to their Class (I,II,II), molecular weight, lipid solubility, and affinity constant in binding to Iron (Fe), Zinc (Zn), and Gallium (Ga).

TABLE I

| | | Abbreviated | Mol. | Lipid | Logβ | Logβ | Logβ |
|---|---|---|---|---|---|---|---|
| Class | Chelator | Name | Wt. | Solubility | —Fe^{III} | —Zn^{II} | —Ga^{II} |
| I | Desferrioxamine | DFO | 656.8 | Low | 30.5 | 11.1 | 27.6 |
| | (Deferoxamine) | | | | | | |
| I | Ethylenediamine- | EDTA | 374.3 | Low | 25.1 | 16.5 | 21.0 |
| | tetraacetic | | | | | | |
| | acid | | | | | | |
| I | Silybin (from | Silybin | 482.4 | Low | 41.5 | — | — |
| | Silibum | | | | | | |
| | marianum) | | | | | | |
| II | Deferiprone | L1 | 141.1 | Moderate | 37.2 | 13.5 | 32.6 |
| | (Dimethyl-3- | | | | | | |
| | hydroxypyrid-4- | | | | | | |
| | one) | | | | | | |
| II | Picolinic acid | Picolinic | 123.1 | High | 12.8 | 12.9 | — |
| | (Pyridine-2- | acid | | | | | |
| | carboxylic acid) | | | | | | |
| III | Dipyridine | BIP | 156.2 | High | 16.3 | 13.2 | 7.7 |
| | (Bypryidine) | | | | | | |
| | (2,2′-bypryidyl) | | | | | | |

Table I illustrates how Class I chelators (EDTA, Silybin) of higher molecular weight remain extracellular because of their lower lipid solubility. Class I chelators are transferred iron/zinc from Class II and III chelators (L1, Picolinic acid, BIP) which enter and leave cells but posses lower affinity for bound iron/zinc. Class III Chelators (BIP), which demonstrate greater affinity for iron/zinc than gallium, are able to carry gallium into cells but then depart from cells with iron/zinc.

In preferred embodiments, certain combinations of Class I, II, and III Chelators in association with one or more cruciferous indoles, e.g., DIM, in topical delivery systems, parenteral delivery systems, oral delivery systems, and simultaneous delivery by multiple routes provides therapeutic efficacy more than the additive efficacy of each agent used alone at maximal dose. Therefore, the methods of combined use at less than maximal dose increase both the safety and efficacy of cruciferous indoles and metal chelators in papillomavirus-related conditions.

Improved efficacy results in a shorter duration of required therapy than with individual agents used alone. Combined use allows a reduction in dose or concentration of each component in topical formulations. Combined use improves the long term therapeutic result with a lower rate of recurrence due to persisting virally infected cells. Combined use with lowered dose and duration of use minimizes toxicity, particularly from the iron/zinc chelators, known to be responsible for systemic toxicity. Combined use with higher dose improves the efficacy of dose-dependent therapy of papillomavirus-related cancer therapy to overcome cancer cell resistance to individual agent therapy alone.

In methods involving the oral use of one or more cruciferous indoles, e.g., DIM, with topical use of one or more cruciferous indoles, e.g., DIM, and an iron/zinc chelator, and, optionally, gallium, the oral delivery of indole is facilitated and accomplished according to formulations and methods described in U.S. Pat. No. 6,086,915, incorporated by reference in its entirety. The new uses of cruciferous indole, e.g., DIM, described here, increase the effectiveness of high-dose DIM and related indoles delivered in various ways for papillomavirus-related conditions and described in pending U.S. patent application Ser. No. 10/117,288, incorporated by reference in its entirety.

The treatment of cutaneous, oral, and genital manifestations of HPV infection with an oral cruciferous indole, e.g., DIM, is facilitated by topical, intravenous, intra-lesional, and aerosol application of cruciferous indoles in specific relative doses to the simultaneous administration of metal chelators. These therapies include production of tinctures, creams, vaginal or rectal suppositories, eye drops, emulsions for intravenous use, and injectable suspensions to deliver synergistic amounts of these agents. The present invention demonstrates an enhanced response in papillomavirus-related cervical cancer cells when one or more cruciferous indoles, e.g., DIM, is used in combination with iron/zinc chelators not seen in earlier reported cell culture studies using DIM alone (Chen DZ, Qi M, Auborn K J and Carter T H, Indole-3-carbinol and diindolylmethane induce apoptosis of human cervical cancer cells and in murine HPV16-transgenic preneoplastic cervical epithelium. J Nutr. 2001 December;131(12):3294-302). Similarly, addition of one or more cruciferous indoles, e.g., DIM, to cell culture of papillomavirus-related cervical cancer cells treated with picolinic acid and other chelators demonstrated a degree of response not seen with picolinic acid alone (see, e.g., U.S. Pat. Nos. 5,767,135 and 6,410,570), or, alternatively seen with the histone deacetylase inhibitors, sodium butyrate or related suberoylanilide hydroxamic acid, alone. A number of one or more cruciferous indoles, e.g., DIM, iron/zinc chelator combinations were found to exceed the action Fernando-Pol (U.S. Pat. No. 6,410,570) demonstrated in cell culture which required 3 millimolar (mM) picolinic acid when used alone. Most effective were combinations that utilized DIM with membrane permeable iron/zinc chelators such as deferiprone. Also highly effective were DIM in combination with chelator mixtures including both a membrane impermeable Class I Chelator such as Silybin combined with a membrane permeable Class II Chelator such as deferiprone or picolinic acid. This approach is presumed to optimize egress of iron and zinc with the Class II Chelator serving as a “shuttle” to remove iron/zinc to the extra cellular space, increasing intracellular iron/zinc depletion. Also highly effective were uses of DIM, a membrane permeable Class III Chelator with appropriate differential affinity towards iron/zinc, and salts of the trace element gallium. This combination demonstrated enhanced apoptosis promotion as compared to DIM and to DIM combined with a Class II or Class III membrane permeable chelator alone. Alternatively, intravenous gallium or gallium isotope can be given with DIM, histone deacetylase inhibitors and/or EGFR antagonists during radiation treatments to overcome resistance of radiation-induced apoptosis of papillomavirus-related cancer cells. In addition to the complementary apoptosis-inducing activity of cruciferous indoles, e.g., DIM, the entry of gallium is facilitated by the presence of certain permeable Class III Chelators. Once intracellular, gallium is released due to the greater affinity of the chelator for iron/zinc. The resulting free intracellular gallium displaces iron and/or zinc from metallo-enzymes leading to the facilitated sequestration of iron and zinc by the iron/zinc chelator and their removal to the extracellular space.

Alternatively, co-administration of oral DIM with the topical combinations of DIM, Class II Chelators, and Class III Chelators with gallium and/or sodium butryate promote even more efficient resolution of cutaneous warts. In young women with warts, only topical preparations are used to avoid undesirable metabolic effects of DIM on estrogen metabolism. The topical formulations of DIM, Class II Chelators, Class III Chelators, zinc-binding agents like sodium butyrate, and gallium salts are formulated in creams and ointments with additional penetration enhancing ingredients. Limonene, or its derivative perillyl alcohol is one such penetration enhancing ingredient. The cream is preferably designed as a moisturizing cosmetic that is formulated to allow application directly to warts. Mannitol may be added to topical formulations to increase their osmotic strength. Additionally, acetaminophen may be added for its contribution to apoptosis and pain relief. Additionally, the addition of therapeutic exposure to ultraviolet light enhances oral and topical use of DIM and chelators in the treatment of common verrucae and oro-genital papillomavirus related lesions.

Alternatively, co-administration of oral DIM alone, with oral Silybin or with oral IRESSA® is used with intravenous Gallium nitrate or Gallium-67 isotope in conjunction with radiation therapy to treat papillomavirus-related cancer. To optimize this treatment approach, a related combined parenteral use of DIM and gallium includes the administration of DIM solubilized in a lipid based emulsion for intravenous use in conjunction with intravenous gallium nitrate just before, during and after radiation therapy treatment sessions to achieve maximal tumor cell content of DIM and gallium during the therapeutic radiation exposure. Alternatively, an intravenous DIM emulsion may be infused along with a zinc-binding histone deacetylase inhibitor like sodium butyrate or SAHA immediately before, during and after radiotherapy.

5.1. Synergistic Anti-papillomavirus Compositions

Compositions for the treatment of papillomavirus-related conditions are provided. The compositions comprise two or, optionally, three or more classes of active ingredients: 1) one or more cruciferous indoles, 2) one or more chelators, 3) optionally, gallium and/or sodium butyrate or related SAHA and 4) EGFR antagonist. When all four classes are to be administered, the composition according to the invention may comprise any combination of two, three or all four together. The compositions can comprise the indole, chelator and, optionally, gallium and/or sodium butyrate and/or an EGFR antagonist, together or singularly. The compositions of the invention can be formulated for systemic or local administration. Furthermore, kits are provided comprising the composition of the invention packaged with instructions for their use, preferably, instructions for practicing a method of the invention.

Compositions of the invention comprise therapeutically effective amounts of one or more cruciferous indoles, e.g., DIM, and one or more iron/zinc chelators (Class I, II, and/or III). In certain embodiments, one or more of the following classes of compounds can be included as an active component: butyric acid related zinc binding compounds (i.e., histone deacetylase inhibitors), Gallium salts, and EGFR antagonists. The cruciferous indoles of the compositions of the invention are selected from the group consisting of I3C, DIM, or active DIM metabolites (e.g., R₅′hydroxy-DIM, R₅′methoxy-DIM, R₅ hydroxy-DIM, R₅ methoxy-DIM, R₅′ R₅ dihydroxy-DIM, R₅′ R₅′dimethoxy-DIM, R₅′ R₄′dihydroxy-DIM, R₅ R₄ dihydroxy-DIM, R₅′methoxy R₄′hydroxy-DIM, and R₅ methoxy R₄ hydroxy-DIM). The iron/zinc chelators of the invention are selected from the group consisting of Desferal (Novartis, Basel, Switzerland), ethylenediametetraacetic acid [EDTA], and ethylenetriaminepentaacetic acid [DTPA], Silybin (3,5,7,-trihydroxy-2-[3-(4-hydroxy-3-methoxyphenil)-2-hydroxymethyl-1,4-benxodioxan-6-il]-chronan-4-one), 1,2-Dimethyl-3-hydroxypyrid-4-one (deferiprone, Ferriprox [D1]), Desferri-Exochelin [DFE 772SM] (Keystone Biomedical, Inc.), Silymarin, N,N′-bis(2-hydroxybenzyl)ethylenediamine-N,N′-diacetic acid (HBED), picolinic acid, 3-hydroxypicolinic acid, and fuscaric acid, dihydroxybenzoic acid (2,2-Dihydroxybenzoic acid), didpyridine, bipryidyl (2,2′-bypryidyl), and its derivative, 2,2′-bipyridyl-6-carbothioamide (BPYTA), 1,10-Phenanthroline, zinc-binding Sodium Butyrate (Butyric acid sodium salt), tributyrin, an orally active butyric acid prodrug, and suberoylanilide hydroxamic acid (SAHA), a butyric acid related compound.

In certain embodiments, the compositions include DIM and Iron/zinc chelators in further combination with Gallium salts (Gallium nitrate or Gallium sulfate) or Butyric acid salts (Sodium butyrate or Calcium butyrate). Combinations of DIM, iron/zinc Chelators of Class I,II,III with or without Gallium/Butyric acid salts can be further combined with penetration enhancers for topical formulations (phosphatidyl choline, Vitamin-E TPGS, terpenes [limonene or perrilyl alcohol], and prepared vehicles such as Aquaphor with or without mannitol. Topical formulations further may use formulations steps including the incorporation of all or a portion of the active ingredients in liposomes. For specialized uses the active components may be formed into the following specifically designed formulations.

5.1.1. Suspensions For Intralesional Injection To Be Used With Common Warts (Verrucae), Oral And Laryngeal Papillomas, Genital And Peri-Anal Papillomas And Warts

Such a suspension consists of microcrystalline DIM (0.2-1% wt/volume) in a suspension of physiologic salts, Iron/zinc Chelators, and pH adjusters. pH adjusters such as NaOH are added to bring the pH to 7.5-8. Preferably the iron/zinc chelators will consist of DFO or L1, or the combination of L1 and picolinic acid or HBED. The iron/zinc chelators are present in a concentration of 0.2-0.8% wt/vol. Alternatively, DIM is mixed with gallium salts (1-2% wt/vol) and/or butyric acid salts (1-2% wt/vol) and a Class II or III chelator which possesses greater affinity for iron/zinc than gallium such as L1 or BIP.

5.1.2. Ointments, Gels, And Creams For Topical Use

Ointments, gels and creams for topical use can be used in the treatment of papillomavirus virus related diseases, e.g., common verrucae (plantar or palmar warts). Typical ointments will suspend microcrystalline DIM or its active metabolites in a petroleum based ointment in association with iron/zinc chelator of Class I, II, III (Desferrioxamine,(Desferal, Novartis, Basel, Switzerland)), ethylenediametetraacetic acid [EDTA], and ethylenetriaminepentaacetic acid [DTPA], Silybin (3,5,7,-trihydroxy-2-[3-(4-hydroxy-3-methoxyphenil)-2-hydroxymethyl-1,4-benxodioxan-6-il]-chronan-4-one), 1,2-Dimethyl-3-hydroxypyrid-4-one (deferiprone, Ferriprox [D1]), Desferri-Exochelin [DFE 772SM] (Keystone Biomedical, Inc.), Silymarin, N,N′-bis(2-hydroxybenzyl)ethylenediamine-N,N′-diacetic acid (HBED), picolinic acid, 3-hydroxypicolinic acid, and Fuscaric acid, didpyridine, bipryidyl (2,2′-bypryidyl), and its derivative, 2,2′-bipyridyl-6-carbothioamide (BPYTA), and 1,10-Phenanthroline). Preferred concentration of chelators are from 0.1-2% wt/wt. Alternatively, DIM (0.2-1% wt/wt) is mixed with gallium salts and/or butyric acid salts (1-2% wt/vol) and a Class II or III chelator which posses greater affinity for iron/zinc than gallium such as HBED, BIP or EDTA. Typical creams will use standard emulsions such as Aquafor etc. pH adjusters such as NaOH are added to bring the pH to 7.5-8. Osmotic agents such as mannitol can be added to increase the osmotic compositions of the formulation. Alternatively, penetration enhancing substances such as Limonene or ethanol (1-2% vol/vol) can be added to the ointment or cream formulation. Safe, apoptosis promoting agents including acetaminophen (1-2% wt/vol), and related para aminophenol derivatives can also be added. Finally, addition of specialized lipids such as ceramide, or its synthetic C2 cerimide derivative, adds further skin penetrating and apoptosis-promoting acitivity.

5.1.3. Vaginal Suppository Or Cream

In certain embodiments of the invention, a vaginal suppository or cream formulation is used in the treatment of vaginal or cervical diseases, such as vaginal or cervical dysplasia. Typical suppositories will suspend microcrystalline DIM or its active metabolites in a petroleum based ointment in association with iron/zinc chelators of Class I, II or III (Desferrioxamine, (Desferal, Novartis, Basel, Switzerland)), ethylenediametetraacetic acid [EDTA], and ethylenetriaminepentaacetic acid [DTPA], Silybin (3,5,7,-trihydroxy-2-[3-(4-hydroxy-3-methoxyphenil)-2-hydroxymethyl-1,4-benxodioxan-6-il]-chronan-4-one), 1,2-Dimethyl-3-hydroxypyrid-4-one (deferiprone, Ferriprox [D1]), Desferri-Exochelin [DFE 772SM] (Keystone Biomedical, Inc.), Silymarin, N,N′-bis(2-hydroxybenzyl)ethylenediamine-N,N′-diacetic acid (HBED), picolinic acid, 3-hydroxypicolinic acid, and Fuscaric acid, didpyridine, bipryidyl (2,2′-bypryidyl), and its derivative, 2,2′-bipyridyl-6-carbothioamide (BPYTA), and 1,10-Phenanthroline), with or without gallium salts and or butyric acid salts. Preferred concentration of chelators are from 0.1-2% wt/wt. Alternatively, DIM is mixed with gallium salts (1-2% wt/vol) and or butyric acid salts (1-2% wt/vol) and a chelator of Class II or III which posses greater affinity for iron/zinc than gallium such as BIP or EDTA. Vaginal creams are similarly formulated in an emulsion with a preferable pH in the 4-6 range and dispensed with a suitable applicator system. A preferred vaginal cream contains the chelator system consisting of dihydroxybenzoic acid(0.5-1% wt/vol), sodium butyrate (1-2% wt/vol) Silybin (0.5-1% wt/vol), acetaminophen (1-2% wt/vol), ceramide (0.5-2% wt/vol), and L1 (0.5-1% wt/vol).

5.1.4. Rectal Suppository Or Ointment To Be Used For Anal And Perianal Dysplasia Or Warts

Typical rectal suppositories will suspend microcrystalline DIM or its active metabolites in a petroleum based ointment in association with iron/zinc chelator of Class I, II, III or gallium salts. Alternatively, DIM is mixed with gallium salts and a chelator of Class III which posses greater affinity for iron/zinc than gallium such as BIP. A preferred rectal suppository contains the chelator system consisting of dihydroxybenzoic acid (0.5-1% wt/vol), sodium butyrate (1-2% wt/vol) Silybin (0.5-1% wt/vol), ceramide (0.5-2% wt/vol), acetaminophen (1-2% wt/vol) and L1 (0.5-1% wt/vol).

5.1.5. Pre Or Post-Coital Vaginal Suppository Or Cream To Be Used Before Or After Intercourse To Prevent Infection With Papillomavirus Between Sex Partners

Typical suppositories will suspend microcrystalline DIM or its active metabolites in a petroleum based ointment in association with iron/zinc chelator or gallium salts. Alternatively, DIM is mixed with gallium salts and a chelator which posses greater affinity for iron/zinc than gallium such as BIP or EDTA. A preferred vaginal cream contains the chelator system consisting of dihydroxybenzoic acid (0.5-1% wt/vol), Silybin (0.5-1% wt/vol), sodium butyrate (1-3% wt/vol) and L1 (0.5-1% wt/vol). Vaginal creams are similarly formulated in an emulsion with a preferable pH in the 4.5-5.0 range and suitable applicator system. Alternatively, such pre or post-coital formulations are made with a spermicide of known activity and compatibility with other ingredients to add contraceptive activity to the anti-papillomavirus therapy. These spermicides may include nonylphenol, nonylphenol ethoxylates as found in Conceptrol (containing 4% nonoxynol-9), or other less irritating spermicide.

5.1.6. Enteric-Coated Oral Formulation For Targeting Esophageal Or Colonic Epithelium To Be Used In The Treatment Of Colonic, Rectal And Anal Dysplasia

In one embodiment, absorption enhanced DIM as described in U.S. Pat. No. 6,086,915, incorporated by reference herein in its entirety, is formulated with Iron/zinc Chelators. Preferably the iron/zinc chelators will consist of DFO or diperidone. Alternatively, DIM is mixed with gallium salts and a chelator which posses greater affinity for iron/zinc than gallium such as BIP or EDTA. This mixture is enteric coated through fluid bed granulation according to a technique that protects the formulation from dissolution and release until peroral transit to the colon or takes place. Alternatively, an oral emulsion is formulated which as a liquid targets the esophageal epithelium.

5.1.7. Aerosol Formulation Of DIM And Iron/Zinc Chelators For Treatment Of Oral, Laryngeal, And Tracheal Papillomavirus Conditions

Typically, aerosol suspensions consist of microsyrstalline DIM (0.01-0.25% wt/wt), Diferiprone (0.15-1% wt/wt), sodium butyrate (1-3% wt/vol) and EDTA (0.15-1% wt/wt) suspended in an acceptable aerosol propellant consisting of chloroflurocarbons. These acceptable propellants include dichlorodifluromethhane, trichlorofluromethhane, with dehydrated alcohol USP or lecithin.

5.1.8. Sterile Ophthalmic emulsion of DIM And Iron/Zinc Chelators for Treatment of Conjunctival Papilloma-virus related conditions

Formulation of DIM, Iron/Chelators, and Sodium Butyrate for ophthalmic use is accomplished through manufacture of an emulsion designed for use as eye drops and for topical therapy of the conjunctiva. The emulsion is used to treat papillomavirus related conjunctival infections alone and in conjunction with oral DIM. In addition the ophthalmic emulsion is used in conjunction with radiation therapy and surgery in the treatment of papillomavirus-related conjunctival cancer. Such an emulsion is packaged in opaque, preservative-free, single use plastic vials/applicators.

A preferred ophthalmic emulsion consists of microcrystalline DIM (0.1-0.3%) (mean particle size 0.25 microns), sodium picolinate (0.25-0.5%), and sodium butyrate (0.5-1.0%) as active ingredients. Alternatively, the ophthalmic emulsion may contain microcrystalline DIM (0.1-0.3%), deferiprone (0.1-0.3%) (L1), and sodium butyrate (0.5-1.0%) as active ingredients.

The composition of a preferred ophthalmic emulsion includes the following per ml: DIM (0.1%), sodium picolinate (0.25%), sodium butryate (0.5%), glycerine, castor oil, polysorbate 80, carbomer 1342, purified water and sodium hydroxide to adjust the pH. Homogenization of these ingredients produces a translucent, homogeneous emulsion with a slightly pink color and with a pH of 6.0 to 7.5. Drops of the emulsion are applied 3 or more times daily to the effected eye. The unit dose vial is inverted a few times to disperse the emulsion before applying to the conjuctiva.

5.1.9. Sterile Intravenous Microemulsions of DIM for Use in Conjunction with Chemotherapy, Radiation Therapy, Combined Chemoradiotherapy, and during Combined use with Chelators and/or Gallium

Stable microemulsions of DIM, designed for intravenous use, were developed to provide a convenient means of administering DIM to achieve high tissue concentrations of DIM quickly and at a predictable time. This use facilitates the combined use of DIM with chemotherapy, radiation therapy, combined chemoradiotherapy, and during use with iron/zinc chelators. Intravenous DIM can be used with topical iron/zinc chelators, with or without gallium or additive chemotherapeutic drugs to synergize with ionizing radiation treating papilloma virus related cancers or with ultra-violet light (UVA, UVB, UVC) therapy treating benign papillomavirus related lesions on skin and oro-genital mucosa. In alternative embodiments, DIM analogues including imidazolelyl-3,3′-diindolylmethane, including nitro substituted imidazolelyl-3,3′-diindolylmethanes and Stanford Research Institute DIM derivative SR13668 can be used The low solubility of DIM in both water and lipid required development of a specialized micro-emulsion that utilized phospholipids to optimize the solubility of DIM and improve the stability of the microemulsion. To prepare the micro-emulsion Ethyl oleate (EO), Phosphatidyl Choline (PC) (from egg yolk), and calcein, were purchased from Sigma-Aldrich, Inc (St. Louis, Mo.). Distearoyl-phosphatidylethanolamin-N-poly(ethyleneglycol) 2000 (DSPE-PEG) was purchased from Avanti Polar Lipids (Alabaster, Ala.).

Using a modification of the method of Yu et al. (Yu W et al., A novel approach to the preparation of injectable emulsions by a spontaneous emulsification process. Int. J. Pharm. 1993; 89:139-146), the microemulsion was manufactured as follows: 160 grams of EO and 60 grams of PC were dissolved in 1 liter pure ethanol. 24 grams of microcrystalline DIM (mean particle size 0.25 micron) was added and dissolved in this “oily phase”. 20 grams of DSPEG-PEG was then dissolved in 500 cc of USP water (Aqueous phase). The oily ethanolic solution (oily phase) with the dissolved DIM was then slowly added into the DSPE-PEG solution (aqueous phase) under moderate magnetic stirring. The aqueous phase immediately turned milky with opalescence as the result of the microemulsion produced. The microemulsion was then subjected to low pressure at 360 mm Hg and maintained at 50° C. The low pressure was used to concentrate the emulsion through removal of the ethanol and a portion of the water. Using an infrared absorption assay to determine the DIM content of the microemulsion, a final concentration of DIM of 7.5 mg/ml was established. Sodium hydroxide was added to increase the pH to the 5.0-7.5 range.

Using this manufacturing technique emulsions of DIM were prepared and subjected to stability testing to demonstrate that the particle size within the emulsion remained between 150 and 200 nm. The production technique resulted in a micro-emulsion with % weight ranges of the components in the following preferred ranges:

| Component | Approx % Weight |
|---|---|
| DIM | .05-.1 |
| Lipids (EO:PC:DSPE-PEG; 8:3:1) | 45-28 |
| Water | 50-70 |
| Ethanol | 1-2 |

Alternatively, an ethanol-free production method can be utilized to produce a stable micro-emulsion of DIM or DIM derivatives and analogues, using Lipofundin MCT B. Braun Melsungen A G (Melsungen, Germany), a preformed basic emulsion, and high pressure homogenization of microcrystalline DIM. This method utilizes jet-milled DIM, with particle size reduced to 0.1 micron average diameter (performed by Micron Technologies, Inc., Exton, Pa.). Using this technique 700 mg of 0.1 micron diameter DIM crystals are homogenized in 100 cc Lipofundin using equipment and methods as described (Akkar A and Muller R H, Formulation of intravenous carbamazepine emulsions by SolEmuls technology. Eur J Pharm Biopharm. 2003 May;55(3):305-12). This results in a stable lipid-based micro-emulsion with particle size less than 200 nm and a DIM content of 7 mg/cc of the emulsion.

5.2. Methods of Treating Papillomavirus-Related Conditions

Papillomavirus-related conditions are treated according to the methods of the invention, which comprise the steps of administering two or, optionally, three or more classes of active ingredients: 1) one or more cruciferous indoles, 2) one or more chelators, and optionally one or more of 3) gallium or gallium salt, 4) an EGFR antagonist, 5) a zinc-binding histone deacetylase inhibitor, or 6) a radiation sensitizing chemotherapeutic. The indoles, chelators and optional ingredients may be administered in any order, or simultaneously. When three or more classes are administered, any combination of two or more may be administered simultaneously, followed by the remaining ingredients. The compositions administered can comprise the indole, iron/zinc chelator and, optionally, gallium and/or an EGFR antagonist, together or singularly. Each of the classes of active ingredients may be administered systemically or locally, or systemically and locally. In addition, therapeutic promotion of apoptosis in papillomavirus-affected skin by indoles and chelators is further enhanced by local irradiation with UVB light at the sites of papillomavirus related lesions using minimal erythemic doses (MED). Non-limiting examples of methods for treating various papillomavirus-related conditions are provided below.

5.2.1. Methods Of Treating Vaginal And Cervical Dysplasia

Cruciferous indoles, e.g., DIM in combination with Class II Chelators, or Class III Chelators and gallium may be administered in the form of a vaginal cream or suppository containing microcrystalline DIM suspended in vitamin-E TPGS (Eastman Company, Kingsport, Tenn.) in a dose of 200-1000 mg/5 cc in combination with dihydroxybenzoic acid (0.5-1% wt/vol), Silybin (0.5-1% wt/vol), and L1 (0.5-1% wt/vol). Alternatively, Silybin (0.5-1% wt/vol), Picolinic acid (3-5% wt/vol) and Sodium Butyrate (3-5% wt/vol) are the chelators. Alternatively, EDTA (3-5% wt/vol) and Picolinic acid (3-5% wt/vol) are the chelators. This allows application of DIM, EDTA, Silybin, L1, sodium butyrate and picolinate directly to vaginal mucosa for enhanced uptake and benefit of genital warts and related vaginal or cervical dysplasia. Alternatively, DIM in a dose of 200-500 mg/5 cc is formulated in combination with a Class I Chelator such as EDTA, and a Class II Chelator such as L1, in a formulation which includes Limonene (1-2%) for penetration enhancement in a vaginal suppository.

Alternatively, DIM in combinations described for vaginal use may be administered in the form of a rectal suppository containing microcrystalline DIM. DIM is suspended in vitamin-E TPGS (Eastman Company, Kingsport, Tenn.) in a dose of 200-1000 mg in combination with Citric acid (0.5-1% wt/vol), Silybin (0.5-1% wt/vol), and L1 (0.5-1% wt/vol. Alternatively, Silybin (0.5-1% wt/vol), Picolinic acid (3-5% wt/vol), and sodium butyrate (2-5% wt/vol). Alternatively, EDTA (3-5% wt/vol) and picolinic acid (3-5% wt/vol) are the chelators. This allows application of DIM, EDTA, Silybin, L1, sodium butyrate and picolinate directly to anal mucosa for enhanced uptake and benefit of genital warts and related anal or rectal dysplasia. Alternatively, DIM in a dose of 200-500 mg/5 cc is formulated in combination with a Class I Chelator such as EDTA, and a Class II Chelator such as L1, in a formulation which includes Limonene (1-2%) for penetration enhancement in a rectal suppository. Alternatively, DIM in a dose of 200-500 mg is formulated in combination with a Class I Chelator such as EDTA, and a Class II Chelator such as L1, in a formulation which includes Limonene for penetration enhancement in a rectal suppository. This allows application of DIM chelator combinations directly to rectal mucosa for enhanced uptake and benefit of peri-anal warts and related anal dysplasia.

Alternatively, DIM for oral use in an absorption-enhanced formulation can be given concomitantly with the topical formulations described in the treatment described for cervical, vaginal, anal or rectal dysplasia. In severe cases, topical irradiation using a standard UVB light source delivering UVB light (Philips TL-01 florescent lamp, emitting UV light at 311 to 312 nm) or other UVB emitting device, is used in addition following oral and topical doses of indoles and chelators to accelerate apoptosis of virally infected skin or mucosal cells. Typically the minimal erythema dose (MED) is determined for skin and then 70% of the MED is delivered to skin or mucosal lesions on a weekly basis. The dose is augmented by 20% each week if tolerated without erythema. UVB light (less than 320 nm) is preferred over UVA light (320-360 nm), since UVB exposure avoids the skin and mucosal immune suppressing effects of UVA radiation.

In addition, the cruciferous indole, e.g., DIM, iron/zinc chelator of Class II and III, and gallium combinations of the present invention may be administered in any appropriate amount in any suitable galenic formulation and following any regime of administration.

The actual administered amount of cruciferous indole, e.g., DIM, iron/zinc chelator of Class I, II, III, gallium, and UVB light combinations may be decided by a supervising physician and may depend on multiple factors, such as, the age, sex, condition, file history, etc., of the patient in question.

The subject, or patient, to be treated using the methods of the invention is an animal, e.g., a mammal, and is preferably human, and can be male or female, child, or adult.

5.2.2. Methods Of Treating Palmar Or Plantar Warts With Combined Formulations Of Cruciferous Indole, e.g., DIM And Chelators

Common verrucae, when present on the hands and feet (Palmar and Plantar Warts), are treated with topical formulations and with intralesional suspensions of cruciferous indole, e.g., DIM combined with iron/chelators. Further synergism for the promotion of apoptosis in papillomavirus infected epidermal cells is promoted with application of ultraviolet (UVB) light inconjunction with topical therapy. Topical therapy in children involves the twice daily application of topical preparations together with oral use of absorption-enhanced DIM at a dose of 2-3 mg/kg/day of DIM (8-12 mg/kg/day of total formula weight of the absorption-enhanced DIM). As described topical preparations preferably consist of microcrystalline DIM, a Class II or III chelator, and gallium salt when a class III chelator is used. Therapy typically lasts 3-4 weeks. Treatment success is documented by the disappearance of warts. This is often associated with temporary hyperpigmentation at the former site of lesions. In adults, the success and rapidity of treatment is increased with the addition of intralesional injections of suspensions of combinations of microcrystalline DIM, iron/zinc chelators, and gallium. Typically, a sterile suspension for such use consists of DIM, sodium butyrate, EDTA, deferiprone, limonene, and ethanol in an aqueous vehicle, as described above. Alternatively, the suspension consists of DIM, dipyridyl, gallium salt, and ethanol in an aqueous vehicle. Subcutaneous intradermal injections using small volumes of 0.2-0.4 ccs of suspension are administered weekly or bi-weekly. Each administration of intradermal DIM is optionally followed by therapeutic irradiation using UVB light. In severe cases, topical irradiation using a standard UV light source delivering UVB light (Philips TL-01 florescent lamp, emitting UVB light at 311 to 312 nm) or other UVB emitting device, is used following oral and topical doses of idoles and chelators to accelerate treatment of resistant warts. Typically the minimal erythema dose (MED) is determined and then 70% of the MED is delivered to skin lesions on a weekly or bi-weekly basis. The dose is augmented by 20% each session if tolerated without erythema.

Topical application of an ointment, cream or gel twice daily improves the success of intralesional therapy. The typical duration of therapy is from 2 to 4 weeks.

5.2.3. Methods Of Treating Recurrent Laryngeal Papillomas Due To Papillomavirus

Recurrent Laryngeal Papillomatosis (RRP) is a rare but debilitating condition effecting a group of children who acquire the human papillomavirus (HPV) on their vocal cords during the birth process from a mother infected with genital HPV. An even smaller group of adults acquire the disease later in life through unknown mechanisms. Periodic surgical excision of recurrent vocal cord papillomas is the standard treatment. While oral doses of absorption-enhanced diindolylmethane (U.S. Pat. No. 6,093,706) have proven of benefit in some cases of RRP, more consistent therapies are needed. The present invention provides for enhanced therapy of RRP with combined preparations of iron/zinc chelators and DIM administered as intralesional injections at the time of surgery, and, through regular topical application of combined formulations in the form of aerosol preparation of microcrystalline DIM and chelators.

Typical intralesional preparations consist of a sterile suspension of microsyrstalline DIM (0.01-0.5% wt/vol), sodium butyrate (1-2% wt/vol) and HBED (1-2% wt/vol) in physiologic saline with PH adjusted to 7-8 with added NaOH and suspension stabilizers. Small volumes of 0.1-0.2 cc of well mixed suspension are injected into tissue forming the base of papillomas following their excision at the time of surgery.

Before and after surgery, an aerosol formulation of cruciferous indole, e.g., DIM and Chelators is applied to the vocal cords and surrounding tissue by inhalation from a metered dose inhaler up to 3 times per day. Additionally, aerosol treatment can be used in conjunction with additional oral cruciferous indole, e.g., DIM, and irradiation with UVB light.

In severe cases, topical irradiation using a specialized UV light source delivering UVB light (emitting UVB light at 311 to 312 nm) or other UVB emitting device allowing irradiation through a fiber-optic laryngoscope is used following oral and topical doses of indoles and chelators to accelerate treatment of oro-pharyngeal, vocal cord, or tracheal papillomas. Typically the minimal erythema dose (MED) is determined and then 70% of the MED is delivered to mucosal lesions on a weekly or bi-weekly basis. The dose is augmented by 20% each session if tolerated without airway compromise due to swelling. Alternatively, 400-800 Joules of UVB per m² corrected for the area to be irradiated can be used as a starting dose.

5.2.4. Methods Of Treating A Male For Asymptomatic Prostatic Infection With The Human Papillomavirus

Recent improvements in documentation of the presence of papillomavirus DNA by polymerase chain reaction (PCR) testing methods have documented the occurrence of asymptomatic prostatic infection with papillomavirus in men (Zambrano A, Kalantari M, Simoneau A, Jensen J L and Villarreal L P, Detection of human polyomaviruses and papillomaviruses in prostatic tissue reveals the prostate as a habitat for multiple viral infections. Prostate. 2002 Dec. 1;53(4):263-76). This asymptomatic condition may be treated in men whose semen samples test positive for papillomavirus using PCR the synergistic combination of cruciferous indole, e.g., DIM and iron/zinc chelators of the present invention. This application involves oral therapy with absorption-enhance DIM in conjunction with oral therapy using, orally active Butyrate (e.g., Tributyrin [Glyceryl tibutyrate, Sigma-Aldrich, St. Louis, Mo.]), orally active silybin (Siliphos, Idena, Inc.) and/or orally active Deferiprone. In this method absorption enhanced-DIM is used at a dose of 3-4 mg/kg day for 3-4 weeks concurrently with oral Deferiprone taken at 25-75 mg/day. Alternatively, the oral DIM can be used with Tributrin, an orally active form of Sodium Butyrate at 50-150 mg/kg/day. Alternatively, the oral DIM can be used in conjunction with the parenteral combined use of EDTA (20-50 mg/kg) and Deferiprone (30-70 mg/kg) administered subcutaneously or intravenously on a weekly basis for 3 weeks. Repeat analysis of a semen sample by PCR for papillomavirus DNA is used to confirm the success of therapy (Rintala M A, Pollanen P P, Nikkanen V P, Grenman S E and Syrjanen S M, Human papillomavirus DNA is found in the vas deferens. J Infect Dis. 2002 Jun 11;185(11):1664-7).

5.2.5. Methods Of Treating A Female To Prevent Transmission Of Papillomavirus In Association With Sexual Intercourse

Using the present invention, the synergistic activity of DIM and iron/zinc chelators can be used to prevent the transmission of papillomavirus from a male carrier to a female recipient at the time of sexual intercourse. This prophylactic use of combined formulations involves the pre-coital application of a vaginal suppository, cream, or gel containing DIM combined with Chelators of Class II, or preferably of Class I and II. Typically the suppository would consist of DIM and Sodium Butyrate. Alternatively, the suppository would include DIM, Tributyrin (Glyceryl tibutyrate, Sigma-Aldrich, St. Louis, Mo.), silybin (Siliphos, Idena, Inc.) and Deferiprone (Apotex Labs, Toronto, Canada). Alternatively, the suppository would consist of DIM, EDTA, Deferiprone, and Picolinic Acid. Additionally, the suppository may contain ascorbic acid as a further chelator, pH adjusters to maintain a suitable pH between 4.5 and 7, and mucosal penetration enhancing compounds such as urea, ceramide, ceramide derivatives (C2 ceramide), and mannitol suitable for vaginal use. The vaginal cream or gel may also be incorporated into a cervical cap or cervical sponge device, or applied to vaginal diaphragm to optimize delivery of medicaments to the cervical mucosa. Additionally, the vaginal suppository, cream, or gel may include a spermicide to add contraceptive activity to the anti-papillomavirus therapy. These spermicides include, but are not limited to, nonylphenol, nonylphenol ethoxylates as found in Conceptrol (containing 4% nonoxynol-9), or other less irritating spermicide. Concurrent use of oral, absorption enhanced DIM in capsules is also used to optimize the intra-vaginal prophylactic therapy when oral doses are taken at least 2 hours before intercourse and consist of at least 2 mg/kg of cruciferous indole, e.g., DIM.

5.2.6. Method Of Treating A Female Following Sexual Intercourse To Prevent Colonization With Papillomavirus

In cases where women are at risk for transmission of papillomavirus following unprotected sexual intercourse, the described vaginal suppositories and creams together with cervical sponges and caps can be used for post-coital therapy. In this case combination preparations using cruciferous indole, e.g., DIM combined with Class I and II chelators are used as once daily intra-vaginal therapy. This may be used in conjunction with oral use of absorption-enhanced DIM at a daily dose of 2 mg/Kg taken as a single daily dose. Success of the therapy is confirmed by cervical smear analyzed for the presence of papillomavirus DNA two weeks or more following therapy by a health care practitioner.

5.2.7. Methods Of Treating Actinic Keratosis And Basal Cell Carcinomas Of The Skin

Actinic keratosis are typical dry, raised, proliferative skin lesions noted more commonly in the elderly and more often in sun exposed skin. Papillomavirus contributes to the occurrence and progression of this common type of skin lesion due to its presence in hair follicles (Majewski S and Jablonska S, Do epidermodysplasia verruciformis human papillomaviruses contribute to malignant and benign epidermal proliferations? Arch Dermatol. 2002 May;138(5):649-54). Actinic keratosis are known to benefit from topical retinoid therapy, but definitive therapy currently requires surgical excision or cryotherapy which are scar forming procedures. Small basal cell carcinoma of the epidermis similarly require surgical excision or cryotherapy.

The formulations of the present invention further provide a means for enhanced topical and non-scarring therapy for actinic keratosis and non-invasive basal cell carcinomas. The formulations can be used in conjunction with topical fluorouracil (USP 5-FU, 1-5%), currently in use for treating these conditions.

This method involves intradermal injection of a sterile suspension of cruciferous indole, e.g., DIM and iron/zinc chelators. The suspension for this use typically consists of microcrystalline DIM and Class I and II chelators. As described above, in one embodiment, this combination consists of DIM, Silybin, and Deferiprone. Alternatively the suspension consists of DIM, Deferiprone and gallium salt. Typically, bi-weekly injections of small volumes of the well-mixed suspension (0.2-0.4 cc) of the sterile suspension subcutaneously in the dermis just below keratoses or basal cell cancers is supplanted by twice daily application of a topical cream or gel. The topical cream or gel consist of a penetration enhanced formula of DIM, iron/zinc chelators of Class III and gallium salts as described above. Typically these ingredients, together with a penetration enhancer such as limonene are added to gel, cream, or ointment base and applied to the effected skin at least twice a day.

In severe cases, each administration of intradermal DIM is optionally followed by therapeutic irradiation using UVB light. In such cases, topical irradiation using a standard UV light source delivering UVB light (Philips TL-01 florescent lamp, emitting UVB light at 311 to 312 nm) or other UVB emitting device, is used following oral and topical doses of indoles and chelators to accelerate treatment of keratosis and non-invasive basal cell cancers. Typically the minimal erythema dose (MED) is determined and then 70% of the MED is delivered to skin lesions on a weekly basis. Normal surrounding skin is protected from UVB by pre-treatment application of 30 SPF or greater topical sunscreen specific for UVB. The dose is augmented by 20% each session if tolerated without erythema.

5.2.8. Method of treating conjunctival papillomavirus-related infection or tumor using combinations of DIM and Iron/Zinc disrupting agents in an opthalmic suspension.

Conjunctival papilloma and ptyerigium, are associated with the presence of papillomavirus DNA in the majority of cases. In addition, a subset of conjunctival squamous cell cancers and lacrimal sack tumors have demonstrated the presence of Papillomavirus DNA. Typically, surgical excision of the conjunctival lesions represent first line treatment, but papillomas frequently recur. Use of an ophthalmic suspension pre- and postoperatively reduces the chance of recurrence.

A preferred ophthalmic emulsion consists of microcrystalline DIM (0.1-0.3%) (mean particle size 0.25 microns), Sodium picolinate (0.25-0.5%), and Sodium Butyrate (0.5-1.0%) as active ingredients. Alternatively, the ophthalmic emulsion may contain microcrystalline DIM (0.1-0.3%), deferiprone (0.1-0.3%) (L1), and Sodium butyrate (0.5-1.0%) as active ingredients. The ophthalmic emulsion is instilled in the effected eye three time a day. In addition, a petrolatum based eye ointment may be utilized with DIM (0.1-0.3%), Sodium picolinate (0.25-0.5%), and Sodium Butyrate (0.5-1.0%) present as active ingredients for use during sleep or while effected eyes are patched closed.

A similar treatment plan using DIM/chelator emulsion and ointment can be used in conjunction with radiation therapy performed for recurrent squamous cell cancer of the conjunctiva. Ideally the treatment would include a means of precisely regulating the site and depth of treatment using “Cyberknife” technology (Accuray, Inc., Sunnyvale, Calif.).

5.2.9. Methods of treating papillomavirus-related cancer using combinations of DIM and Iron/Zinc chelators in conjunction with Radiation Therapy and EGF receptor antagonists

Cancers of the upper aerodigestive tract are known to be associated with the presence of papillomaviruses. These cancers include head and neck tumors (cancers of the oral cavity, pharynx, and larynx) and certain esophageal cancers. Advanced tumors involving the base of the tongue and tonsillar fossae are rarely cured even by radical surgery and radiation therapy and carry a poor prognosis. The combination of radiation therapy with standard chemotherapy improves therapeutic response, but is still associated with serious side effects from each modality. Despite attempts to maximize the radiation dose and optimally fractionate total dose, no one treatment plan has proven superior in head and neck cancer (Mendenhall W M, Morris C G, Amdur R J, Hinerman R W, Mancuso A A. Parameters that predict local control after definitive radiotherapy for squamous cell carcinoma of the head and neck. Head Neck. 2003 July;25(7):535-42). Technology for optimizing radiation therapy is taught in U.S. Pat. No. 6,477,229, hereby expressly incorporated by reference in its entirety. Therapeutic techniques which allow a reduction in radiation dose and improved response to radiation due to co-administered radiosensitizing agents are needed.

Often, in patients with locally invasive and metastatic disease, surgical resection is not possible and these patients are treated with primary Radiation Therapy and Chemotherapy to reduce the size of the tumor mass. This therapy is only palliative and typically tumors develop resistance to both the action of ionizing radiation and chemotherapy drugs. Methods of improving the success of non-surgical treatment have been developed using DIM and Iron/Zinc disrupting agents.

Tumors of the head and neck and esophagus are known to concentrate Gallium. Injection of Gallium-67 (Ga-67) isotope with Scintography (SPECT Imaging) is used to identify the location and spread of such tumors. Since intravenous gallium concentrates in these types of tumor tissue and other forms of squamous cell carcinoma, the pro-apototic action of DIM combined with Gallium, with or without additional Iron/Zinc chelators and chemotherapy agents, can be used in conjunction with Radiation Therapy to improve the efficacy of this mode of cancer treatment.

This method involves oral DIM use combined with intravenous gallium-67 isotope administration, or, simultaneous intravenous use of a DIM emulsion along with intravenous administration of gallium nitrate begun before, continued during, and immediately after the Radiation Therapy session. Typically an oral dose of 2.5-7.5 mg/kg of DIM given orally in an absorption enhanced formulation is given 2 hours before the Radiation Therapy session and continued every 8 hours for 24 hours following a radiation therapy treatment session. Other DIM analogues retaining the apoptosis-promoting activity of DIM may be substituted for oral or intravenous DIM. Intravenous Gallium Nitrate or Ga-67 isotope is begun at least 30 minutes before the treatment session. The Ga-67 isotope is given at a dose of 7-9 millicuries and the Gallium nitrate at a dose of 100 mg/square meter of body surface/day. The Gallium nitrate is diluted in 1000 cc of 0.9% sodium chloride. Starting 30 minutes before radiation therapy, the infusion of Gallium nitrate is continued for about 1 hour after the radiation therapy treatment. Treatments are administered for 5 consecutive days and repeated monthly if required.

Typically, the combined use of DIM and Gallium with Radiation therapy allows a reduction in the total radiation dose and fewer radiation associated side effects including skin changes, dysphagia, and mucositis. Reductions of at least 30% from the typical maximal radiation dose of 7000 cGy for head and neck cancers are possible with this combined therapy. Reduced fractionation of the total radiation dose with fewer treatment sessions is also made possible.

Alternatively, oral DIM alone or in combination with Iron/Zinc chelators can be combined with other orally active chemotherapeutic agents which add to the radiosensitizing effects of DIM and chelators. This particularly applies to the addition of oral Iressa® (Gefitinib [ZD1839]), and other inhibitors of the epidermal growth factor receptor. Other inhibitors of EGF receptor include CI 1033 [Parke-Davis Pharmaceutical Research (Ann Arbor, Mich.)], a quinazoline tyrosine kinase inhibitor different from Iressa, and PKI 166 [Novartis Pharma, AG (Basel)], a non-quinazoline EGFR antagonist. In combined therapy with DIM, ZD1839 is administered orally in a dose of 250-750 mg/day at the same time as oral or intravenous dose of DIM (2.5-10 mg/kg/day) and at least 2 hours prior to a radiation therapy treatment. Both oral ZD1839 and DIM are continued on a three times a day basis during a typical 7 week long series of radiation therapy treatments. Ideally, “Gammaknife” or “Cyberknife” (Accuray, Inc., Sunnyvale, Calif.) radiation therapy technology is also used to concentrate and focus the radiation beam limiting the radiation exposure of normal tissue adjacent and distant to the tumor mass.

5.3. Pharmaceutical Compositions

The pharmaceutical compositions according to the present invention preferably comprise one or more pharmaceutically acceptable carriers and the active constituents. The carrier(s) must be “acceptable” in the sense of being compatible with the other ingredients of the composition and not deleterious to the recipient thereof.

It will be appreciated that the amounts of Diindolylmethane or other cruciferous indole, Iron/Zinc chelators, and optionally, gallium and/or EGFR antagonist, required for said treatments will vary according to the route of administration, the severity of the papillomavirus-related disease, age, and file history of the subject, the galenic formulation of the pharmaceutical composition, etc.

Preferably, the diindolylmethane used in the invention has been processed to enhance bioavailability, as is described in United States Patent Application No. 6,086,915, incorporated herein by reference in its entirety; however any suitable preparation of pure diidolylmethane can be used in the methods and compositions of the invention.

In general, a suitable (therapeutically effective) amount of Diindolylmethane is preferably administered in an absorption enhancing formulation, as described in United States Patent Application No. 6,086,915, at 150-750 mg per day as a suspension of microparticles in a starch carrier matrix. The actually administered amounts of Diindolylmethane may be decided by a supervising physician. The Diindolylmethane of the invention may be administered in combination with Iron/zinc chelators, gallium, sodium butyrate, or EGFR antagonist administered by either oral, topical, or parenteral routes.

Therapeutic formulations include those suitable for parenteral (including intramuscular and intravenous), topical, oral, rectal or intradermal administration, although oral administration for DIM is the preferred route. Thus, the pharmaceutical composition may be formulated as tablets, pills, syrups, capsules, suppositories, ophthalmic suspension, formulations for transdermal application, powders, especially lyophilized powders for reconstitution with a carrier for intravenous administration, etc.

The term “carrier” refers to a diluent, adjuvant, excipient, or vehicle with which the therapeutic is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin; and/or a flavoring agent, such as peppermint, methyl salicylate, or orange flavoring.

Therapeutic formulations suitable for oral administration, e.g., tablets and pills, may be obtained by compression or molding, optionally with one or more accessory ingredients. Compressed tablets may be prepared by mixing phytochemicals, and compressing this mixture in a suitable apparatus into tablets having a suitable size. Prior to the mixing, the indole or orally active chelator may be mixed with a binder, a lubricant, an inert diluent and/or a disintegrating agent.

In a preferred embodiment, Diindolylmethane is mixed with a binder, such as microcrystalline cellulose, and a surfactant, such as sodium lauryl sulphate until a homogeneous mixture is obtained. Subsequently, another binder, such as polyvidone, is transferred to the mixture under stirring with a small amount of added water. This mixture is passed through granulating sieves and dried by desiccation before compression into tablets in a standard tableting apparatus.

A tablet may be coated or uncoated. An uncoated tablet may be scored. A coated tablet may be coated with sugar, shellac, film or other enteric coating agents.

Therapeutic formulations suitable for parenteral administration include sterile solutions or suspensions of the active constituents. An aqueous or oily carrier may be used. Such pharmaceutical carriers can be sterile liquids, such as water and oils, including those of petroleum, animal, vegetable or synthetic origin, such as peanut oil, soybean oil, mineral oil, sesame oil and the like. Formulations for parenteral administration also include a lyophilized powder comprising phytochemical that is to be reconstituted by dissolving in a pharmaceutically acceptable carrier that dissolves said phytochemical. Parenteral administration also includes a stable emulsion of DIM designed for intravenous use. Ideally, the emulsion prevents the early removal of DIM from the circulation due to early uptake by the reticuloendothelial system allowing maximal cellular concentration of DIM in papillomavirus infected cells or tumor tissue.

When the pharmaceutical composition is a capsule, it may contain a liquid carrier, such as a fatty oil, e.g., cacao butter.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. The composition can be formulated as a suppository, with traditional binders and carriers such as triglycerides.

In yet another embodiment, the therapeutic compound can be delivered in a controlled release system. In one embodiment, a pump may be used (see Langer, supra; Sefton, CRC Crit. Ref. Biomed. Eng. 1987, 14:201; Buchwald et al., Surgery 1980, 88:507; Saudek et al., N. Engl. J. Med. 1989, 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 1983, 23:61; see also Levy et al., Science 1985, 228:190; During et al., Ann. Neurol. 1989, 25:351; Howard et al., J. Neurosurg. 1989, 71:105).

Other controlled release systems are discussed in the review by Langer (Science 249:1527-1533 (1990)).

In one embodiment of the pharmaceutical composition according to the invention, the Diindolylmethane, PREG, and DHEA are comprised as separate entities. The three entities may be administered simultaneously or sequentially.

The invention also provides a pharmaceutical pack or kit comprising one or more containers filled with one or more of the ingredients of the pharmaceutical compositions of the invention. This includes the combination of capsules for oral use and creams or gels for simultaneous topical application. Optionally associated with such container(s) can be a notice in the form prescribed by a governmental agency regulating the manufacture, use or sale of pharmaceuticals or biological products, which notice reflects approval by the agency of manufacture, use or sale for human administration.

A number of references have been cited, the entire disclosures of which are incorporated herein by reference.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

### 6. EXAMPLES

### 6.1. Example

### Synergistic Promotion Of Apoptosis By DIM, And Iron/Zinc Disruptors Demonstrated In Cultured Papillomavirus Transformed Cells

In vitro cell culture experiments were performed to investigate the induction of apoptosis by combinations of Diindolylmethane (DIM), Silybin (SY), Picolinic Acid (PA), Sodium Butyrate (BA), and Gallium Nitrate (Ga) in relevant cervical cancer cells. The cell lines CaSki (containing multiple copies of integrated HPV16 DNA), C33A (HPV negative with mutant p53), were utilized. Cells were maintained and assays for apoptosis induction performed as previously described (Chen D Z, Qi M, Auborn K J and Carter T H, Indole-3-carbinol and diindolylmethane induce apoptosis of human cervical cancer cells and in murine HPV16-transgenic preneoplastic cervical epithelium. J Nutr. 2001 December;131(12):3294-302). Novel combinations of DIM and various Iron/Zinc chelators were tested to assess and document greater than additive (synergistic) apoptosis-related activity in controlled 72 hour cultures.

The primary assay for apoptosis-related loss of cell viability was the mitochondrial function assay [reduction of 3-(4,5-dimethylthiazol-2-yl)-5-(3-carboxymethoxyphenyl)-2-(4-sulfophenyl)-2H-tetrazolium (MTS)] using a MTS kit (Promega, Madison, Wis.). A minimum of 4 replicate wells per condition were studied and absorbance at 595 nm of the solution in individual wells was determined with a multi-well plate reader. Data were analyzed by plotting the mean and SD of cell viability versus concentration of DIM, Iron/Zinc chelator or other growth inhibitors.

A secondary assay to confirm the apoptoic mechanism of cell death measuring nucleosomal leakage was utilized. This assay detects histones and DNA in cytoplasmic extract and utilizes Cell Death Detection ELISA Plus kit from Roche Molecular Biochemical (Mannheim, Germany). Results were determined by measuring absorbance at 405 nm with the multi-well plate reader.

Agents Tested:

Diindolylmethane (DIM): microcrystalline from BioResponse, LLC

Silybin (SY): crystalline, from LKT, Labs

Gallium Nitrate (Ga): crystalline, from Alfa Aesar

Butryric acid (Bu): crystalline, from Sigma

Picolinic acid (Pa): crystalline, from Sigma

Deferiprone: crystalline from Apotex Labs

Desferioxamine: lyophilized, from Sigma

Results:

Reproducible curves for cell viability reduction for each agent used alone were established, together with dosage for 50% inhibition of cell viability (ID₅₀) for individual agents in 72 hr cultures.

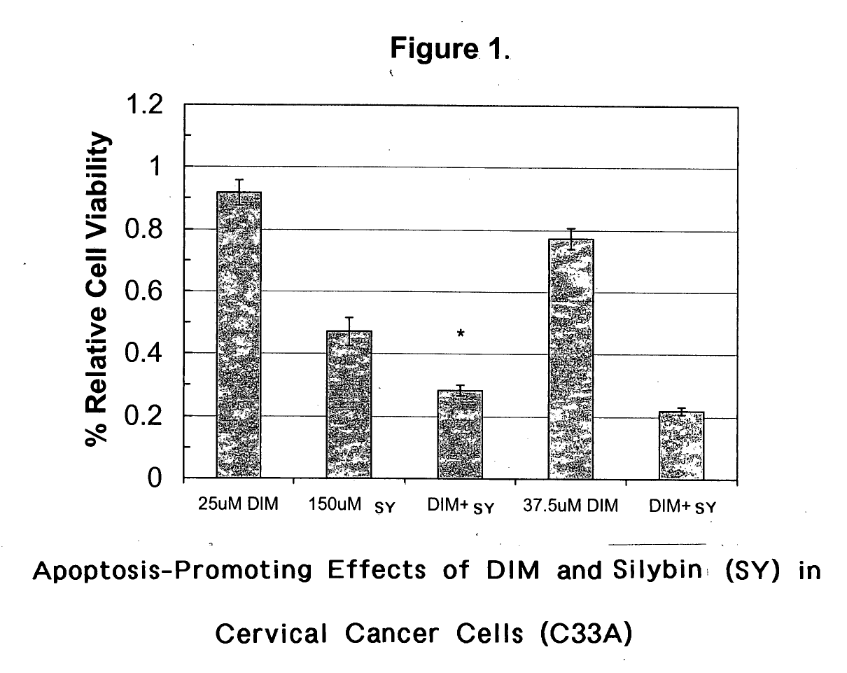

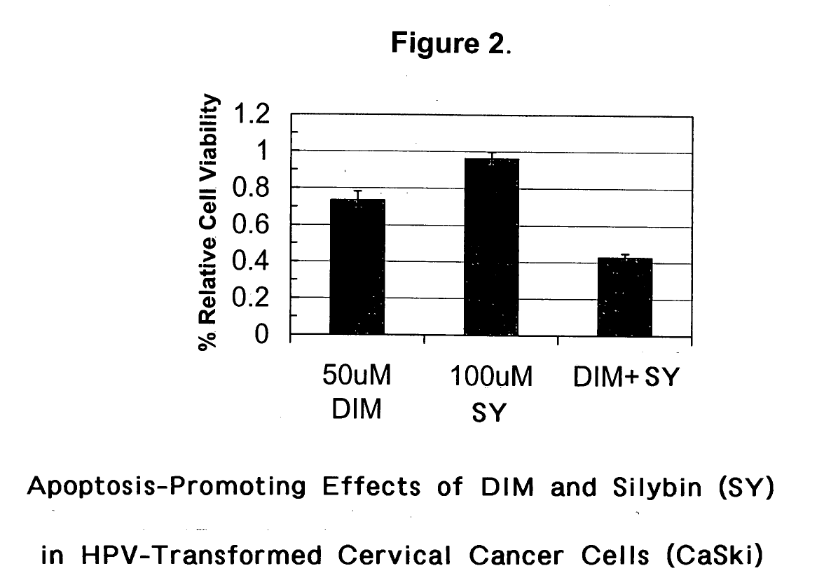

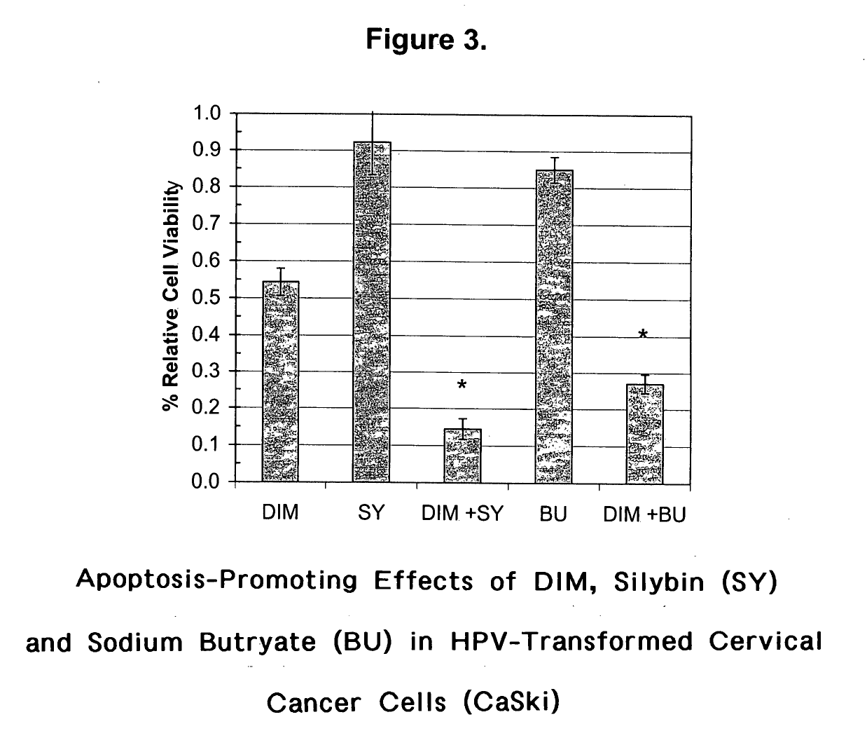

Using doses for each agent at or below the ID₅₀ levels, synergistic, apoptosis-related, reductions in cell viability were documented for both combinations of DIM and Iron disrupting compounds and for combinations of DIM and Zinc disrupting compounds. Results showing percent reductions of cell viability using the MTS assay for individual agents, combined effects, and extent of synergism are summarized in the following tables and FIGS. 1-3.

TABLE II
DIM Combined with an Iron Chelator

| | | | | | | | PerCent |
|---|---|---|---|---|---|---|---|
| | | DIM | | SY | Expected | Observed | Synergistic |
| Cell | DIM | Effect | SY | Effect | Combined | Combined | Increase in |
| Line | Dose | Alone | Dose | Alone | Effect | Effect | Combined Effect |
| C33A | 25 μM | −8% | 150 μM | −54% | −62% | −72% | 16.6% |
| CaSki | 50 μM | −26% | 150 μM | −4% | −30% | −57% | 56.6% |
| CaSki | 75 μM | −46% | 150 μM | −8% | −54% | −86% | 59.2% |

TABLE III
DIM Combined with an Iron/Zinc Chelator

| | | | | | Expected | Observed | PerCent |
|---|---|---|---|---|---|---|---|
| | | DIM | | Pa | Combined | Combined | Synergistic |
| Cell | DIM | Effect | Pa | Effect | Effect DIM | Effect DIM | Increase in |
| Line | Dose | Alone | Dose | Alone | plus Pa | plus Pa | Combined Effect |
| CaSki | 75 μM | −19% | 2.5 mM | −13% | −32% | −41% | 28.1% |
| CaSki | 100 μM | −21% | 2.5 mM | −13% | −34% | −75% | 120.5% |

TABLE IV
DIM Combined with a Zinc Chelator

| | | DIM | | BU | Expected | Observed | Synergistic |
|---|---|---|---|---|---|---|---|
| Cell | DIM | Effect | BU | Effect | Combined | Combined | Increase in |
| Line | Dose | Alone | Dose | Alone | Effect | Effect | Combined Effect |
| CaSki | 75 μM | −46% | 150 μM | −15% | −61% | −73% | 18.2% |

TABLE V
DIM and Iron/Zinc Chelator Combinations

| | DIM | Sy | Sy and DIM | Sy and DIM | PerCent | Sy 150 μM | Sy, Bu, and | Sy, Bu, and | PerCent Dim |
|---|---|---|---|---|---|---|---|---|---|
| Cell | Alone | Alone | Expected | Observed | Synergy | and Bu | DIM Expected | DIM Observed | Sy, and |
| Line | 75 μM | 150 μM | Effect | Effect | DIM and Sy | 3 mM Alone | Effect | Effect | Bu Synergy |
| CaSki | −19% | −5% | −21% | −45% | 114% | −1% | −20% | −69% | 245% |

Conclusions:

A synergistic increase in apoptotic cell death in two relevant cell lines for papillomavirus-related infection and papilloma-virus-related cancer was shown to result from the combined treatment of cells with DIM and Iron/Zinc chelating compounds. Confirmation that the observed effect was due to promotion of apoptosis and not due to separate mitochondrial or other unanticipated cell toxicity was provided by use of the secondary nucleosomal leakage assay. These results provide a basis for the clinical benefits of DIM and Iron/Zinc disrupting compounds when used together in methods for treating papilloma-virus related conditions, as shown in the following examples.

### 6.2. Example

### In vivo Demonstration Of Synergistic Action Of DIM-Iron/Zinc Chelator Formulations In Patients With Common, Recalcitrant Warts

An adult subject with multiple recurrent warts on both feet was recruited and treated as follows. The subject received two tubes of ointment labeled A and B without knowing their contents. Tube A contained the active ingredients DIM and Limonene without a chelator in a penetration enhanced vehicle. Tube B contained DIM, the chelators L1, picolinic acid, sodium butyrate and Limonene in the same penetration enhancing vehicle. The subject was instructed to apply contents of tube A to warts on the right foot only. Hands were then washed and the contents of tube B was applied to warts on the left foot only. Before treatment photographs of the feet were obtained by a collaborating podiatrist. Subject was instructed to apply ointments as directed twice a day. Re-evaluation at 2 weeks revealed improvement in warts treated with the contents of Tube B (DIM plus Iron/zinc chelators and sodium butyrate) and no significant change in warts treated with the contents of tube A (DIM alone). The subject was then instructed to dispose of tube A and begin treating the unchanged warts on the right foot with contents of tube C. Tube C contained Iron Chelator (Picolinic acid and sodium butyrate) and Limonene without DIM in a penetration enhanced vehicle. When re-examined after one month of treatment the warts on the right foot treated sequentially with the contents of Tube A (DIM alone) and Tube C (Iron/zinc Chelator alone) showed minimal improvement. The warts on the left foot treated with the contents of tube B(DIM plus Iron/zinc Chelator) showed complete resolution.

### 6.3. Example

### In vivo Demonstration Of Synergistic Action Of DIM-Iron/zinc Chelator-Gallium Formulations In Patients With Common, Recalcitrant Warts

A subject with multiple recurrent warts on both feet is recruited and undergoes treatment as follows. The warts on both feet are injected intradermally below the warts with a solution of 2% gallium nitrate in saline. The subject is then treated as follows: The subject receives two tubes of ointment labeled A and B without knowing their contents. Tube A contains Iron Chelator (L1) and Limonene without DIM in a penetration enhancing vehicle. Tube B contains DIM in combination with L1 (iron chelator) and Limonene in the same penetration enhancing vehicle. The subject applies contents of tube A to warts on the right foot only. Hands are then washed and the contents of tube B is applied to warts on the left foot only. Before treatment, photographs of the feet are obtained by the collaborating podiatrist. The subject is instructed to apply ointments as directed twice a day. The subject is re-evaluated at 1 week for improvement in warts treated with the contents of Tube B (DIM plus Iron Chelator) and warts treated with the contents of tube A (Iron Chelator alone). Intradermal gallium injection is repeated for warts on both feet. The subject is then instructed to continue treatment for one additional week. The subject is re-examined after three full weeks of treatment.

### 6.4. Example

### In vivo Demonstration Of Synergistic Action Of DIM and Iron/zinc Chelators In K14-HPV16 Transgenic Mice As Compared To Treatment With DIM Alone

Transgenic mice expressing the human HPV E6 and E7 oncogenes under control of the keratin 14 promoter all develop cervical cancer when exposed chronically to estradiol. Applying the methods of Jin et al. (Jin L. et al., Indole-3-carbinol prevents cervical cancer in human papilloma virus type 16 (HPV16) transgenic mice, Cancer Research 1999, 59(16):3991-7) the K14-HPV16 transgenic mouse model is employed to demonstrate the therapeutic advantage of combined oral treatment with DIM and iron/zinc chelators in vivo. This in vivo model utilizes K14-HPV16 mice maintained and fed on AIN76a diet as described (Chen DZ, Qi M, Auborn KJ, Carter TH. Indole-3-carbinol and diindolylmethane induce apoptosis of human cervical cancer cells and in murine HPV16-transgenic preneoplastic cervical epithelium. J Nutr. 2001 December;131(12):3294-302). Virgin, 4-5 week old, female K14-HPV16 mice are divided into groups of 20 animals and housed 5 animals per cage. All animals are implanted subcutaneously with 0.25 mg/day release pellets of Estradiol, and implants are repeated every 60 days until the end of the study. Mice are maintained on experimental diets as described below until 24 weeks of age. Then, following euthanasia, the vagina, cervix and both uterine horns are removed and fixed in 10% formalin in PBS. The cervical tissue for each animal is subsequently examined using the following methods able to detect the presence of cervical cancer as well as cellular and molecular markers of induced apoptotic activity:

1. Cervical tissue sections are stained with Hemotoxylin and Eosin stain and examined by light microscopy.

2. Tissue assay for activated caspase. Cervical tissue slices are fixed and processed for immunostaining as described (Chen D Z, Qi M, Auborn K J and Carter T H, Indole-3-carbinol and diindolylmethane induce apoptosis of human cervical cancer cells and in murine HPV16-transgenic preneoplastic cervical epithelium. J Nutr. 2001 December;131(12):3294-302). The tissue slices are incubated with a polyclonal antibody specific for the activated form of caspase 3 (Promega) overnight. Tissue slices are then incubated with a peroxidase-goat anti-rabbit second antibody (Santa Cruz Biochemicals) and the immunofluorescence for each tissue sample is quantified. The level of caspase 3 activation serves as a molecular marker of apoptotic activity.

3. Tissue assay for cell fraction undergoing apoptosis. A TdT-mediated dUTP nick end labeling (TUNEL) assay is used to stain and assess tissue sections for the number of cervical cells showing evidence of apoptosis (Complete ApopTag in situ hybridization kit [Intergen, Purchase, NY]). TUNEL staining of each cervical sample is scored by a single individual unaware of the treatment groups.

A summary of animal groups and treatments are as follows:

1. Control Diet plus (placebo pellet)

2. Control Diet plus 0.250 mg/day estradiol pellet

3. Estradiol pellet, plus DIM Treatment Diet(DIM): (Control Diet plus DIM 20 mg/kg/day [BioResponse-DIM, BioResponse LLC, Boulder Colo.]

4. Estradiol pellet, plus Silybin Treatment Diet (SY): (Control Diet plus Silybin 100 mg/kg/day [Silybin from SiliPhos®, [Indena, Inc., #1 dB 1016])

5. Estradiol pellet, plus Tributyrin Treatment Diet(BU): (Control Diet plus Butyrate from butanoic acid, 1,2,3-propanetriyl ester (Tributryin), at Butyrate 100 mg/kg/day from Tributyrin (Sigma, St Louis, Mo.)

6. Estradiol pellet, plus DIM plus Silybin Treatment diet (DIM-SY) (Control Diet plus 20 mg/kg/day DIM, plus (Silybin 100 mg/kg/day [Silybin from SiliPhos®, [Indena, Inc., #1 dB 1016])

7. Estradiol pellet, plus DIM and Butyrate Treatment Diet (DIM-BU) (Control Diet plus 20 mg/kg/day DIM, plus Butyrate from butanoic acid, 1,2,3-propanetriyl ester (Tributryin), at Butyrate 100 mg/kg/day from Tributyrin (Sigma, St Louis, Mo.)

8. Estradiol pellet, plus DIM and Butyrate Treatment Diet (DIM-BU) (Control Diet plus 20 mg/kg/day DIM, plus Butyrate from butanoic acid, 1,2,3-propanetriyl ester (Tributryin), at Butyrate 50 mg/kg/day from Tributyrin (Sigma, St Louis, Mo.) and Silybin 50 mg/kg/day [Silybin from SiliPhos®, [Indena, Inc., #IdB 1016])

After 18 weeks of treatment, the mice are sacrificed and examined for cervical tumors. Results are compared for rates of apoptosis detected by examining and scoring the cervical epithelium by the methods described.

### 6.5. Example

### Manufacture Of Processed DIM For Enhanced Oral Bioavailability

Preparation of processed Diindolylmethane was accomplished according to the steps outlined in United States Patent Application No. 6,086,915, herein incorporated by reference in its entirety. Briefly, this included mixture of about 10-40% by final weight of either Diindolylmethane with about 10-40% by final weight of vitamin E polyethylene glycol 1000 succinate (Vitamin-E-TPGS, Eastman Chemical), 2-20% by final weight, phosphatidyl choline (Phospholipon 50G, Rhone Poulenc) and 15-30% by final weight hexanol. This mixture was made homogeneous by mixing. The homogeneous mixture of indoles and other oil soluble substituents listed above was added to a solution of modified starch in water (Capsul Starch from National Starch, Inc.). The starch component forms from 30-70% of the final dry weight of the product. The well dispersed final combined mixture was then subjected to spray drying. The resultant product was a fine powder containing either Diindolylmethane contained within the starch particles.

### 6.6. Example

### Manufacture Of Capsules Containing Diindolylmethane

Capsules containing 150-300 mg of processed Diindolylmethane, as produced according to the steps described in example 6.5, were made by mixing the processed Diindolylmethane with microcrystaline cellulose and placing the mixed powder into opaque gelatin capsules.

### 6.7. Example

### Manufacture Of DIM With An Iron/zinc Chelator In A Cream For Transdermal Delivery

For the aqueous phase of the emulsion, a mixture of 70 grams of propylene glycol, 15 grams of Picolinic acid (2-Picolinic acid, Sigma Chemicals, P5503), 15 grams of sodium butyrate (Aldrich 303410) and 633 grams of water was heated to 95° C. The oil phase of the emulsion was prepared by heating a mixture of the following to 105° C.: 30 grams cetostearyl alcohol (Alfol 16/18, Vista), 30 grams hydrogenated soy monoglyceride (Myverol 18-06, Quest), 30 g of a mixture of polyoxyethylene stearic acid ester and mono- and di-glycerides of fatty acids (Arlacel 165, ICI), 10 grams polyethylene (Epolene N-34, Eastman), and 50 g of squalene. The active ingredient phase was prepared separately also by gently heating to about 63° C. a mixture of the following to uniformity: 30 g d-Alpha-tocopherol polyethylene glycol 1000 succinate (Vitamin E TPGS, Eastman), 50 g isopropyl myristate, 7.5 g of DIM (LKT Labs, St. Paul, Minn.), and 7.5 g Silybin (LKT Labs, St. Paul, Minn.). The above oil phase was added to the aqueous phase using a rotor/stator type homogenizer at moderate speed. The mixture was cooled to 75° C. and 50 grams of lemon oil is added with low speed mixing followed by addition of the active ingredient phase. Lastly, 2 g of a 3:1 mixture of methyl paraben to propyl paraben was added to the emulsion. This mixture was transferred to the reservoir of a high pressure homogenizer such as the Microfluidics Model 110Y. The emulsion was passed through the homogenizer approximately five times at 15,000 psi operating pressure that is sufficient to form a cream of the desired consistency which will not separate on standing. Alternatively, the cream was produced with 15 grams of 1,2-Dimethyl-3-hydroxypyrid-4-one (deferiprone, Ferriprox, Apotex Labs, Canada) replacing the Picolinic acid in the aqueous phase.

Alternatively, the transdermal preparation would include Sodium Butyrate (2-4% wt/vol) as the sodium salt or as Tributyrin (Glyceryl tibutyrate, butanoic acid, 1,2,3-propanetriyl ester, Sigma-Aldrich, St. Louis, Mo.), or 2-Furildioxime (4-5% wt/vol) (DFO, Eastman Kodak, Rochester, N.Y.), alone or together with ceramide or synthetic ceramide derivatives, C2 ceramide (2-4% wt/vol), and additional ethanol to serve as co-solvent and penetration enhancer.

### 6.8. Example

### Manufacture Of DIM With Iron/Chelator In A Suppository for Vaginal or Rectal Administration

In a heated vessel, 90 grams cetostearyl alcohol (Alfol 16/18, Vista) mixed with 10 cc Grapfruit Oil (Aldrich Chemical) was heated to 100 Degrees C. to which 5 gms of microcrystalline DIM, 10 gms of Silybin (LKT Labs, St. Paul, Minn.), and 10 gms of deferiprone, Ferriprox, Apotex Labs, Canada) were added with constant mixing to form a hot slurry. Alternatively, 90 grams cetostearyl alcohol (Alfol 16/18, Vista) is heated to 100 Degrees C. to which 5 gms of microcrystalline DIM is mixed and to which is added 10 grams of Tributyrin (Glyceryl tibutyrate, Sigma-Aldrich, St. Louis, Mo.), alone or together with 10 grams of ceramide or synthetic cerimide derivatives, C2 ceramide. In a second vessel 400 gms of IV Novata (Semi-synthetic Glyceride Suppository Base, Ashland Chemicals) was warmed to 40 Degrees C. with constant mixing. The well mixed slurry from the first vessel was added with continued mixing to the second vessel. The homogenized molted suppository material was formed into suppositories of 2 gms each and cooled. Glyceryl monsterate 10-50 gms was added to the molten mixture as needed to increase the firmness of the final suppositories.

### 6.9. Example

### Manufacture Of DIM With Iron/zinc Chelators In A Penetrating Oil for Topical Administration.

In a heated vessel, 500 cc of Grapefruit Oil (a source of concentrated Limonene) (Aldrich Chemical) was heated to 50 Degrees C. to which to which 7.5 gms of microcrystalline DIM, 10 gms of Siybin (LKT Labs, St. Paul, Minn.), and 25 gms of 15 grams of Picolinic acid (2-Picolinic acid, Sigma Chemicals, P5503) were added with constant mixing. The mixture was cooled and transferred to 10 cc brown glass bottles equipped with glass-rod applicator tops. The Penetrating DIM-Chelator Oil was applied 2-3 times per day directly to warts on the hands and feet.

Alternatively, sodium butyrate or deferiprone, (Ferriprox, Apotex Labs, Canada) is utilized in place of the Picolinic acid in the penetrating oil.

### 6.10. Example

### Combined Oral And Transdermal Use Of Diindolylmethane In Combination With Iron/zinc Chelators For The Treatment Of Plantar Warts In A Child

Plantar warts, or verrucae involving the soles of the feet, are a particularly difficult variety of verrucae to successfully treat. Surgical ablation in addition to topical caustic treatment of the underlying dermis revealed at surgery is typically required for long-term eradication. The contribution of dietary supplementation with the cruciferous phytochemical, DIM in association with an Iron/zinc chelator in a DIM topical formulation in treating plantar warts (verruca vulgaris) is illustrated by the following case history.

A. L., a 14 year old adolescent girl with recurrent plantar warts involving the soles of both feet, was the subject of this study. She presented to the podiatrist having failed to respond to topical Aldara Cream. Pretreatment photos of her feet were obtained and she was started on a combined oral and topical treatment plan. She began taking a daily dose of 10 mg/kg absorption enhanced DIM formulation (2.5 mg/kg of actual DIM) taken in capsules as a twice daily dose. She was dispensed a bottle of penetrating oil containing DIM, Silybin, sodium butyrate, Picolinic acid, and Grapefruit oil (See Example 6.9). The oil was applied to plantar warts at least twice a day and additionally after showering or bathing. Follow up by the treating Podiatrist at 1 month revealed disappearance of all warts. There was return of normal skin lines and some residual hyperpigmentation over the former location of some of the larger warts.

### 6.11. Example

### Combined Oral And Transdermal Use Of Diindolylmethane In Combination With Iron/zinc Chelators For The Treatment Of Plantar Warts in an Adult.

S. C., a 48 year woman with multiple palmar warts involving both hands, is the subject of this study. She suffers from rheumatoid arthritis which is treated with low dose methotrexate and periodic oral steroids (prednisone). Cryotherapy of the warts and Alara cream treatments were unsuccessful at removing her warts. An alternative therapy was sought.

Oral, absorption enhanced DIM formulation was begun at 5 mg/kg per day, taken as a single once daily dose with breakfast. A sterile suspension of microcrystalline DIM, Deferiprone, and gallium nitrate was prepared by a compounding pharmacist (See Section 5.1.1). Using a 25 gauge needle, the attending physician injected approximately 0.1-0.2 cc 1% Xylocaine below each lesion using a 27 gauge needle and syringe. Following this, the physician used a 23 gauge needle and syringe to inject approximately 0.1-0.2 cc of well mixed DIM-chelator-gallium suspension just below each of the locally anesthetized warts. Then, topical irradiation using a standard UV light source delivering UVB light (Philips TL-01 florescent lamp, emitting UVB light at 311 to 312 nm) was used following topical doses of indoles and chelators. The patient was dispensed a hand cream formulated with DIM, Silybin, Deferiprone, and Limonene as active ingredients (See Example 6.7). The patient applied the hand cream 2-3 time a day and additionally after bathing. The UVB irradiation was repeated after 1 week. She returned for follow-up at two weeks and demonstrated shrinkage in her wart lesions. Some had assumed a dark brown to black color. A minority of the larger lesions were re-injected with the suspension according to the described procedure. The hand cream and oral DIM capsules were continued for two additional weeks. Follow-up at 1 month after starting treatment revealed disappearance of all warts and return of normal skin lines. There was no change in arthritic symptoms or levels of rheumatoid factor determined by blood test during treatment.

### 6.12. Example

### Combined DIM and Intravenous Gallium Nitrate treatment in a patient with oropharyngeal cancer to improve response to Radiation Therapy

Currently, radiation, surgical or combined radiation and surgical treatment of oropharyngeal squamous cell carcinoma involving the tonsilar fosssa carries about a 50% risk of local recurrence in larger, stage T₃ tumors (greater than 4 cm in greatest dimension) or locally advanced tumors involving multiple regional lymph nodes. This recurrence rate remains the same with and without the additional surgical implantation of radioactive seeds (Brachytherapy), or additional chemotherapy. With current therapy, typical 4 year survival rates are less than 50%. Standard radiation therapy involves 6000 to 7000 cGy total radiation dose, fractionated over a 6-8 week treatment schedule.

Side effects from primary radiation therapy of oropharyngeal cancer are common and related to the radiaton dose. The most debilitating are oropharyneal mucositis and moderate to severe dysphagia. These cause malnutrition, compromise patient survival, and often require drastic support measures like gastrostomy and intravenous hyperalimentation to overcome. Additionally, xerostomia (dry mouth) and loss of taste are expected side effects.

A 59 year old male diagnosed with a T3, N0, squamous cell cancer of the right tosilar fossa elected to add indole and chelator therapy to the primary radiation therapy for his tumor. Fixation of his glottis and early extention of the tumor to the base of the tongue made him a poor surgical candidate.

In order to reduce radiation-related side effects use of both conventional radiation and Cyberknife radiosurgery (Accuray, Sunnyvale, Calif.) were employed together with intravenous and oral DIM and intravenous Ga-67 isotope and Gallium nitrate. This combined approach allowed reduction of the total radiation dose from 7000 cGy to 3500 cGy. On the first treatment day the patient underwent Computerized Tomography (CT) with intravenous omnipaque to enhance 3 dimensional definition of the tumor mass. This was followed by a Ga-67 SPECT scan involving administration of 8 millicuries of Gallium-67 Citrate isotope (Cardinal Health, Denver, Colo.). This scan documented preferential uptake of Gallium by the tumor tissue.

On the afternoon of the first treatment day, the patient underwent initial radiation therapy preceded by an intravenous DIM infusion one hour before treatment and a Gallium nitrate infusion before and during treatment. The Gallium nitrate was given at a dose of 100 mg/square meter of body surface/day, diluted in 1000 cc of 0.9% sodium chloride. A 700 cGy radiation treatment was then delivered by the Cyberknife. Following this, oral DIM was continued every 8 hours at a dose of 2.5 mg/kg of DIM. The one week treatment course for the patient is summarized in the following chart:

TABLE VI

| Treatment | | | | | |
|---|---|---|---|---|---|
| Component | Day 1 | Day 2 | Day 3 | Day 4 | Day 5 |
| CT with | X | | | | |
| I.V. Contrast | | | | | |
| Gallium-67 | X | | X | | |
| I.V. with Scan | | | | | |
| I.V. Gallium | X | | X | | X |
| Nitrate | | | | | |
| I.V. DIM | X | | X | | X |
| Infusion | | | | | |
| Cyberknife | X | X | X | X | X |
| Radiation Tx | | | | | |
| Post-Radiation | X | X | X | X | X |
| Oral DIM Capsules | | | | | |

### 6.13. Example

### Combined Gallium-67, Oral Dim, and Oral Iressa® Treatment in a Patient with Oropharyngeal Cancer to Overcome Cancer Cell Resistance to Radiation Therapy

A 60-year-old female patient suffered a local recurrence at the original site of a T2, NO tonsilar squamous cell carcinoma of the left tonsilar fossa. This occurred one year following standard radiation therapy to the primary tumor area with a total dose of 5500 cGy. Following initial therapy the patient had experienced one month of mucositis, dysphagia, and weight loss. She elected Cyberknife only for re-treatment with radiation therapy with the addition of Gallium-67 and combined oral use of DIM and Iressa® (Gefitinib [ZD1839], Astra Zenaca, UK) to help overcome expected radio-resistance of her recurrent tumor and to minimize radiation associated side effects.

On the first treatment day the patient, weighing 50 kg, underwent Computerized Tomography (CT) with intravenous omnipaque to enhance the 3 dimensional definition of the recurrent tumor mass. This was followed by a Ga-67 SPECT scan involving administration of 8 millicuries of Gallium-67 isotope (Cardinal Health, Denver, Colo.). This scan documented preferential uptake of Gallium by the tumor tissue.

On the afternoon of the first treatment day, the patient underwent initial radiation therapy preceded by an oral dose of Iressa® 500 mg (two 250 mg capsules) and oral DIM given at 6 mg/kg (four 75 mg DIM Capsules). Each oral agent was given with 8 oz of water on an empty stomach. 2 hours after the oral doses of DIM and Iressa®, a 500-cGy radiation treatment was delivered to the tumor site by the Cyberknife. Following this oral DIM (four 75 mg DIM capsules) and Iressa® (250 mg per dose) were continued every 8 hours. The one-week treatment course for the patient is summarized in the following chart:

TABLE VII

| Treatment | | | | | |
|---|---|---|---|---|---|
| Component | Day 1 | Day 2 | Day 3 | Day 4 | Day 5 |
| CT with | X | | | | |
| I.V. Contrast | | | | | |
| Gallium-67 | X | | X | | X |
| I.V. with Scan | | | | | |
| Pre-Radiation | X | X | X | X | X |
| Oral Iressa | | | | | |
| 500 mg | | | | | |
| Pre-Radiation | X | X | X | X | X |
| Oral DIM | | | | | |
| 300 mg | | | | | |
| Cyberknife | X | X | X | X | X |
| Radiation Tx | | | | | |
| Post-Radiation | X | X | X | X | X |
| Oral DIM | | | | | |
| 150 mg q 8 hrs | | | | | |
| Post-Radiation | X | X | X | X | X |
| Oral Iressa | | | | | |
| 250 mg q 8 hrs | | | | | |

The reduced total radiation dose through use of the Cyberknife is expected to reduce severe mucositis and dysphagia. The specific activities of DIM and Iressa® interact to inhibit anti-apoptotic tumor cell mechanisms of radio-resistance in the squamous cell carcinoma.

### 6.14. Example

### Use of Primary Cultures of Human Tumors to Demonstrate Synergistic Apoptosis Promotion in vitro with the Combination of DIM, Iron/Zinc Chelators and/or Iressa®.

A protocol to establish the synergistic activity of DIM, selected Iron/Zinc chelators, and/or Gefitinib (Iressa®, ZD1839 [Astra Zeneca]) based on the exposure of primary cultures of human tumors is designed. Iressa® is an orally active EGFR-TKI (epidermal growth factor receptor tyrosine kinase inhibitor) which blocks signal transduction pathways which may contribute to chemotherapy and radiation resistant cancer. Other inhibitors of the epidermal growth factor receptor (EGFR) to be tested include CI 1033 [Parke-Davis Pharmaceutical Research (Ann Arbor, Mich.)], a quinazoline tyrosine kinase inhibitor different from Iressa, and PKI 166 [Novartis Pharma, AG (Basel)], a non-quinazoline EGFR antagonist. The effects of DIM alone and in combination on tumor growth are evaluated using the EVA/PCD (ex vivo apoptotic/programmed cell death) assay (Rational Therapeutics Cancer Evaluation Laboratories, Long Beach, Calif.) which has previously been shown to correlate with response, time to progression and survival in patients.

Serial dilutions of DIM alone and in combination with Zinc-binding deacetylase inhibitors, Iron chelators, and Iressa® are applied to biopsy specimens of non-small-cell lung cancer (NSCLC), breast, colon, and prostate cancers. The PIP kinase inhibitor wortmannin in combination with DIM and other agents is also used to assess the influence of agents on the Akt-related pathway of apoptosis. Dose-response curves are interpolated to provide 50% lethal concentrations (LC(50)). The degree of synergy (by median effect) and normalised Z-scores (raw scores converted to relative activity distributed around the mean) is then computed.

Favorable interactions are anticipated for DIM combinations with EGF receptor antagonists. Tumor cultures will be analyzed for synergistic increases in apoptosis-related cell killing with combinations of DIM and EGF inhibitors, DIM and Zinc-binding histone deacetylase inhibitors (HDAC's), and with the combination of DIM, HDAC's, and EGF Inhibitors.

These primary human culture studies may support synergistic and possibly clinically beneficial interactions of DIM, EGF inhibitors, and Iron/Zinc Chelators.

What is claimed is:

1. A method of treating a papillomavirus related epithelial disorder comprising administering to a subject in need thereof a therapeutically effective amount of one or more iron/zinc chelators and one or more cruciferous indoles.

2. The method of claim 1, where the one or more chelators and one or more indoles are administered simultaneously.

3. The method of claim 1, wherein the one or more chelators and one or more indoles are administered within a short time of one another.

4. The method of claim 1, wherein the one or more indoles are administered orally.

5. The method of claim 1, wherein the one or more iron/zinc chelators and one or more cruciferous indoles are administered topically.

6. The method of claim 1, wherein the amount of the one or more indoles is lower than that which is therapeutically effective when the one or more indoles are administered in the absence of the one or more chelators.

7. The method of claim 1, wherein the amount of the one or more chelators is lower than that which is therapeutically effective when the one or more chelators are administered in the absence of the one or more indoles.

8. The method of claim 6, wherein the amount of the one or more chelators is lower than that which is therapeutically effective when the one or more chelators are administered in the absence of the one or more indoles.

9. The method of claim 1 wherein the one or more chelators and the one or more indoles act synergistically.

10. The method of claim 1, further comprising the administration of a therapeutically effective amount of one or more compounds selected from the group consisting of gallium, a gallium salt, a zinc-binding histone deacetylase inhibitor and an EGFR antagonist.

11. The method of claim 1, further comprising the administration of a therapeutically effective amount of gallium or a gallium salt.

12. The method of claim 11, wherein said gallium is gallium-67.

13. The method of claim 11, wherein the one or more chelators have an affinity for gallium and an affinity for iron/zinc, and wherein the affinity for gallium is less than the affinity for iron/zinc.

14. The method of claim 1 where the one or more indoles are selected from the group consisting of Diindolylmethane (DIM), hydroxy-DIMs, methoxy-DIMs, imidazolelyl-3,3′-diindolylmethane, nitro substituted imidazolelyl-3,31-diindolylmethanes, 2-hydroxy estrogens, and 2-methoxy estrogens.

15. The method of claim 1 wherein the one or more chelators are selected from the group consisting of Desferrioxamine (DFO), 3,5,7,-trihydroxy-2-[3-(4-hydroxy-3-methoxyphenil)-2-hydroxymethyl-1,4-benxodioxan-6-il]-chronan-4-one (Silybin), ethylenediametetraacetic acid [EDTA], ethylenetriaminepentaacetic acid [DTPA], 1,2-Dimethyl-3-hydroxypyrid-4-one (deferiprone, Ferriprox [L1]), Desferri-Exochelin [DFE 772 SM], N,N′-bis(2-hydroxybenzyl)ethylenediamine-N,N′-diacetic acid (HBED), picolinic acid, 3-hydroxypicolinic acid, Fuscaric acid, 2,2′-bypryidyl (dipyridine [bipryidyl]), 2,2′-bipyridyl-6-carbothioamide (BPYTA), 1,10-Phenanthroline and sodium butyrate.

16. The method of claim 1 wherein the papillomavirus related epithelial disorder is selected from the group consisting of oral-genital human papilloma virus infection, oropharyngeal human papilloma virus-related papillomas and dysplasia, peri-anal human papilloma virus-related papilloma and dysplasia, vaginal human papilloma virus-related papilloma and dysplasia, uterine cervical human papilloma virus-related papilloma and dysplasia, skin-related human papilloma virus infection (warts or verrucae), human papilloma virus-related cancer, basal cell carinoma of the skin, carcinoma of the uterine cervix, carcinoma of the uterine endometrium, and carcinoma of the colon.

17. The method of claim 1 wherein the papillomavirus related epithelial disorder is an human papilloma virus-related opthalmic infection.

18. The method of claim 1 or 10 further comprising administering a radiation therapy regimen sufficient to treat a papillomavirus-related disease.

19. The method of claim 18 wherein said radiation therapy comprises topical irradiation with ultraviolet radiation or x-rays.

20. A pharmaceutical composition comprising a therapeutically effective amount of the combination of one or more iron/zinc chelators and one or more cruciferous indoles.

21. The composition of claim 20, wherein the composition is formulated for oral administration.

22. The composition of claim 20, wherein the amount of the one or more indoles is lower than that which is therapeutically effective when the one or more indoles are administered in the absence of the one or more chelators.

23. The composition of claim 20, wherein the amount of the one or more chelators is lower than that which is therapeutically effective when the one or more chelators are administered in the absence of the one or more indoles.

24. The composition of claim 22, wherein the amount of the one or more chelators is lower than that which is therapeutically effective when the one or more chelators are administered in the absence of one or more indoles.

25. The composition of claim 20 wherein the combination is synergistic.

26. The composition of claim 20, further comprising a therapeutically effective amount of one or more compounds selected from the group consisting of gallium a gallium salt, a zinc-binding histone deacetylase inhibitor and an EGFR antagonist

27. The composition of claim 20, further comprising a therapeutically effective amount of gallium or a gallium salt.

28. The composition of claim 27, wherein said gallium is gallium-67.

29. The composition of claim 27, wherein the one or more chelators have an affinity for gallium and an affinity for iron/zinc, and wherein the affinity for gallium is less than the affinity for iron/zinc.

30. The composition of claim 20, wherein the one or more indoles are selected from the group consisting of Diindolylmethane (DIM), hydroxy-DIMs, methoxy-DIMs, imidazolelyl-3,3′-diindolylmethane, nitro substituted imidazolelyl-3,3′-diindolylmethanes, 2-hydroxy estrogens, and 2-methoxy estrogens.

31. The composition of claim 20 wherein the one or more chelators are selected from the group consisting of Desferrioxamine (DFO), 3,5,7,-trihydroxy-2-[3-(4-hydroxy-3-methoxyphenil)-2-hydroxymethyl-1,4-benxodioxan-6-il]-chronan-4-one (Silybin), ethylenediametetraacetic acid [EDTA], ethylenetriaminepentaacetic acid [DTPA], 1,2-Dimethyl-3-hydroxypyrid-4-one (deferiprone, Ferriprox [L1]), Desferri-Exochelin [DFE 772SM], N,N′-bis(2-hydroxybenzyl)ethylenediamine-N,N′-diacetic acid (HBED), picolinic acid, 3-hydroxypicolinic acid, Fuscaric acid, 2,2′-bypryidyl (dipyridine [bipryidyl]), 2,2′-bipyridyl-6-carbothioamide (BPYTA), 1,10-Phenanthroline and sodium butyrate.
